(19) United States
(12) Patent Application Publication
(10) Pub. No.: US 2007/0015179 A1
(43) Pub. Date: Jan. 18, 2007

(54) Plastic microfluidic chip and methods for isolation of nucleic acids from biological samples

(75) Inventors: Catherine M. Klapperich, Boston, MA (US); Arpita Bhattacharyya, Brighton, MA (US)

Correspondence Address: RONALD I. EISENSTEIN, 100 SUMMER STREET, NIXON PEABODY LLP, BOSTON, MA, 02110, US

(73) Assignee: Trustees of Boston University, Boston, MA (US)

(21) Appl. No.: 11/411,528

(22) Filed: Apr. 26, 2006

Related U.S. Application Data
(60) Provisional application No. 60/674,833, filed on Apr. 26, 2005.
(60) Provisional application No. 60/760,691, filed on Jan. 20, 2006.

(51) Int. Cl. C12Q 1/68; C12P 19/34; C12M 3/00

(52) U.S. Cl. 435/6; 435/91.2; 435/287.2; 977/924

(57) ABSTRACT

The present invention is directed to methods of manufacture of microfluidic chip such as a plastic microfluidic chips, which has channels packed with polymer-embedded particles and uses thereof. The chip of the present invention is designed for application of an untreated biological sample on the chip thus allowing isolation, purification and detection of biomolecules, such as nucleic acids, proteins or peptides in one step. The invention also provides a microfluidic chip for combined isolation, purification and detection of biomolecules thus providing a complete Lab-on-a-Chip analysis system for biomolecules such as nucleic acids and proteins. The chips of the invention can be adapted to perform highly specific immunoassays and diagnostic test, for example, for diagnosis of infectious agents, such as bacteria, viruses or parasites.

### CROSS-REFERENCE TO RELATED APPLICATIONS

This application claims benefit of U.S. provisional applications Ser. No. 60/674,833, filed Apr. 26, 2005, and 60/760,691, filed Jan. 20, 2006, the contents of which are herein incorporated by reference in its entirety.

### BACKGROUND OF THE INVENTION

1. Field of the Invention

The present invention relates to a device and methods for their manufacture as well as isolation, purification and detection of biological molecules, such as nucleic acids and proteins. Specifically, the invention relates to the preparation of microfluidic chips that comprise polymer-embedded particles and methods for solid-phase isolation, purification and detection, of biological molecules using such microfluidic chips. Immunoassays and diagnostic tests, for example for detecting microorganisms, such as bacteria, using the device are also provided.

2. Description of the Related Art

The extraction and detection of biomolecules, such as nucleic acids and proteins, from cells, including eukaryotic and prokaryotic cells, is a vital step in many biological and diagnostic applications. Hence, there has been a growing interest in integrating the cell lysis and purification processes of these biomolecules on chip-based microfluidic devices. Such a device would allow higher throughput, lower sample/reagent consumption and significant cost reduction.

Most current microfluidic chip-based devices are made by photolithographic patterning of silicon or glass, or with polydimethylsiloxane (PDMS) using the methods of multilayer soft-lithography. Silicon and glass fabrication can be very expensive, while PDMS lacks dimensional stability and has limited shelf-life. These limitations necessitate the use of alternative materials to make disposable, point-of-care devices, for example, for diagnostic applications. Polymer-based microfluidic chips have been described in the art, for example, U.S. Patent Application No. 2004/0101442. This application described formation of surface-modified microfluidic devices, wherein the microchannel surfaces are physically altered to increase surface area and can be chemically altered to provide additional features. However, this device is not suitable for purification and isolation of nucleic acids.

Microfluidic approaches to DNA purification have been previously demonstrated in glass microchips fabricated by Deep Reactive Ion Etching (DRIE). Recovery of DNA molecules was achieved by packing microchannels with silica particles and immobilizing by a sol-gel method. Currently used methods use nickel alloy molds made with LIGA or electroforming in hot embossing micro scale features into polymeric substrates. These methods are very expensive.

The methods used for monolith formation and attaching the solid phase to the walls of the micro channels employ heating a slurry of tetraethylortho-silicate (TEOS), ethanol and silica particles, a monolith that is covalently attached to the walls of the glass microchip is achieved. However, the sol-gel chemistry involves high temperatures and is not suitable for in situ applications of the polymeric devices.

In existing DNA isolation techniques, cells are typically lysed outside the microchip with conventional methods before the on-chip experiment, and microliters of the cell lysate or purified DNA sample is loaded onto the chip for DNA isolation. Such methods are difficult to implement in other than full diagnostic laboratory settings. This prevents them from being used for, for example critical bacterial strain detection when analyzing causative agents for infections.

Problems also exist with conventional immunoassays. They often require long assay times; require difficult fluid handling techniques and use of relatively large quantity of sample material and reagents. These problems again prevent these assays from becoming a point-of-care diagnostic technique.

For example, for many infectious diseases, effective treatments are available. Getting the correct treatment to a patient quickly is often hindered by the time necessary to confirm a preliminary diagnosis with a laboratory test. The benefits of a speedy diagnosis are obvious, as for example, in the case of a biological attack. More immediately, the ability to differentially diagnose patients in a hospital or nursing home setting will eliminate many unnecessary measures that are often taken to prevent the putative spread of an unspecified infection. In remote or low income areas, the ability to provide laboratory test results during the course of an office visit would greatly reduce the spread of infectious disease and the number of times a patient has to visit the clinic. In all of these cases, the impact on financial and public health costs is significant.

For example, current diagnostic methods for bacterial infections typically require time and a full scale diagnostic laboratory. For some infectious diarrheas, stool cultures have limited clinical utility. Instead, infection is established by a stool bioassay for cytotoxins that cause rounding of cultured fibroblasts (cells from a cell line) or immunoassays for the stool toxins themselves. The cytotoxicity bioassay is considered the gold standard against which other cytotoxin assays are compared, given its high sensitivity (94-100%) and specificity (99%). In this bioassay, stool is diluted with a buffer, filtered to remove bacteria and solids, and then placed in a cultured monolayer of fibroblasts. Toxins produced by the organisms disrupt the cytoskeleton and, when present at levels as low as a few molecules per cell, will cause rounding. The specificity of this cytopathic effect is confirmed by preincubating a control sample with antibodies that neutralize the toxins. Cell rounding not thus blocked is referred to as “nonspecific cytotoxicity” which occurs in only −1% of samples. The bioassay is reported as “positive” or “negative;” titers are not reported as they have no utility. Drawbacks of the cytotoxicity assay are its labor-intensive nature, attendant high cost, and the 48-72 hrs it typically takes to complete.

More rapid assays with reasonable published sensitivity (70-90%) and specificity (99%) are afforded by enzyme linked immunosorbent assays (ELISAs) for some bacterial toxins. Because they are easier to perform, most clinical laboratories have replaced the cytotoxicity bioassay with ELISAs. However, different institutions using different commercially-available kits often experience lower sensitivity and specificity levels during real-world use. Indeed, as a rule, because of its high sensitivity, the cytotoxicity bioassay will consistently detect at least 5-10% of cases missed by ELISA testing.

An example of a difficult to diagnose infectious agent is Clostridium difficile. The spectrum of disease caused by C. difficile infection is broad, ranging from acute watery diarrhea with abdominal pain, low grade fever, and leukocytosis to the major complications of dehydration, hypotension, toxic megacolon, septicemia perforation, and death. Typically, C. difficile-associated diarrhea occurs in elderly hospitalized patients following antibiotic treatment; it is debilitating, and prolongs hospitalization. Recently, cases of the infection have been documented in patients outside of the usual affected groups: younger people and people not in a hospital or institutional environment. This development has been a great cause of concern in the medical community as new strains appear to cause a more severe disease. Distinguish ling C. difficile from other less serious infections with similar symptoms at onset is critical to effective patient care.

Accordingly, it would be highly desirable to develop a device and a method such as a microfluidic chip which would allow application of an untreated biological sample on the chip and result in isolated and purified nucleic acids in one step. Such chips would allow not only purification but also detection and analysis of nucleic acid or protein samples in a so-called “Lab-on-a-Chip” system, i.e., to perform a complete nucleic acid analysis on one single disposable inexpensive microfluidic chip, which would require no additional sample preparation methods, no highly skilled laboratory personnel or expensive laboratory space, and which would use a very small amount of sample and reagent material and result in rapid detection and/or isolation of one or more biological molecules in a sample.

### SUMMARY OF THE INVENTION

The present invention is directed to methods of manufacture of microfluidic chips such as plastic microfluidic chips, which has channels packed with polymer-embedded particles and uses thereof. The chip of the present invention is designed for application of an untreated biological sample on the chip thus allowing isolation, purification and detection of biomolecules, such as nucleic acids or proteins or peptides in one step. The invention also provides a microfluidic chip for combined isolation, purification and detection of biomolecules thus providing a complete Lab-on-a-Chip analysis system for biomolecules such as nucleic acids and proteins. The chips of the invention can be adapted to isolate and/or purify biomolecules, and perform highly specific immunoassays and diagnostic test, for example, for diagnosis of disease causing and/or infectious agents, such as bacteria, viruses or parasites.

For example, the microfluidic immunoassay as described herein offers significant advantages, such as, improved reaction kinetics, multistage automation potential, possibility for parallel processing of multiple analytes, and improved detection limits due to high surface area-to-volume ratio. The immunoassay “lab-on-a-chip” devices and methods of the invention are portable. Accordingly, the device provides an ideal point-of-care diagnostic system.

The invention is based upon a discovery that one can use a porous polymer monolith to embed particles, such as silica-based particles, into a polymer matrix. Photopolymerization of monolith embedded with silica particles is a surprising alternative to the widely-used silica bead/sol-gel approach. In the U.S. Patent Application No. 2004/0101442, Siachowiak et al. demonstrated the formation of polymer monolith inside of a cyclic olefin polymer, wherein the channel walls are modified by a polymer photografting method to encourage formation of covalent bonds with the monolith and prevent formation of voids between the channel wall and the porous monolith. However, the use of the polymer monolith to entrap silica particles as shown by the present invention, has not been previously shown

Here we describe a method of trapping silica particles in a porous polymer monolith to form a solid-phase extraction system. The monolith was formed by in-situ UV polymerization of a monomer mixture impregnated with the silica particles. The high UV transmission of for example, ZEONOR makes it suitable for in-situ photopolymerization applications. We used photoinitiated polymerization prior to the formation of the monolith. The grafted interlayer polymer covalently attaches to the monolith and prevents the formation of voids between the monolith and the channel surface. The interlayer also stops the monolith from migrating down the channel during separations. The porous monolithic columns embedded with silica particles were then used for nucleic acid extraction studies.

The device of the invention is a sample preparation device which is useful in isolating and detecting biomolecules, such as nucleic acids, antibodies, other proteins or peptides, from biological samples via an on-chip solid-phase extraction column, and elution and storage of the isolated nucleic acids on-chip for downstream separation and detection tasks. The device also allows successful extraction and elution of the biomolecules. In contrast to the methods in the prior art, which require separate cell lysis before biomolecule purification, the present device allows cell lysis, for example, with chaotropic agents, thus providing a one-step isolation and purification method for biomolecules.

Accordingly, in one embodiment, the invention provides a microfluidic device comprising: (a) a substrate that is not glass with at least one channel of less than 150 μm in diameter, wherein the channel has an inlet, an outlet, and an internal space with a surface between the inlet and the outlet; (b) a first porous polymer monolith comprising a first monomer within the internal space, wherein the porous polymer monolith comprises a second monomer, and is attached to said first polymer in at least one region of the internal space, wherein the first and the second monomers may be of the same or different material; and (c) a second porous polymer monolith impregnated with particles within said internal space.

The closed chip minimizes the possibility of contamination of the sample by the environment or contamination of the environment by the sample, both important considerations in biological sample preparation and handling. The chip also allows isolation and purification of, for example, nucleic acids from real-world biological samples, and their injection into a holding reservoir, wherein they can be stored for further analysis.

The solid-phase microfluidic chip allows extraction of any kinds of nucleic acids, including naturally occurring, synthetic and modified, DNA and RNA. The particles can also be designed to bind other biomolecules such as antibodies, peptides, and proteins.

For example, in isolation methods from cellular material, a subsequent digestion steps can be used to obtain pure sample of only DNA or RNA. Purified nucleic acids can be easily aspirated from this reservoir. Alternatively, nucleic acid amplification, digestion, sequencing and other detection enhancing methods can be used by providing sufficient reagents, such as enzymes, buffers, primers, and nucleotides, into the reservoir. The reservoir can also be fitted into a thermocycler, for amplification and/or quantification of the nucleic acids using, for example, the PCR technique. Additionally, a detection step may be added to the system allowing detection of the biomolecules, such as cellular or bacterial antigens.

In one embodiment, the invention provides a polymer microfluidic chip with polymer-embedded silicone beads, comprising a polymer matrix with at least one channel

In another embodiment, the invention provides a method of making a microfluidic chip impregnated with porous polymer comprising particles, comprising the steps of providing a polymer micro-chip with at least one channel, photografting the channel by filling the channel with an aromatic ketone, preferably benzophenone and diacrylate solution, irradiating the micro-chip, filling the photografted channel with a polymer solution impregnated with particles, irradiating the polymer-particle mixture thereby forming a microfluidic chip impregnated with porous polymer comprising particles.

The plastic microchips or microfluidic devices described here include a sample preparation module for extraction of nucleic acids from patient samples. Extraction/purification of nucleic acids is a vital step is a number of applications, such as in methods using of nucleic acid probes for genomic DNA in the detection of human pathogens. Thus the plastic chip can function as a portable disease surveillance device. The chip can also be used for isolation of mRNA, to measure gene expression in infected cells or to determine the relative toxicity of a bacterial infection. The chip also provides an ideal purification system for high-speed, high-throughput DNA sequence analysis or other genomic application.

The proposed microfluidic solid phase extraction method will have advantage over the existing technologies in that a chip-based sample preparation system will shrink the conventional “bench-top” “macroscale” procedure into a miniature, portable device. The chips also significantly reduce the sample/reagent consumption. The chips allow purification of nucleic acids from small numbers of mammalian or bacterial cells and thus allow one to process many different samples in parallel. Sample contamination can be significantly minimized by carrying out the procedures in a closed system. Since the chips are made of plastic, they will be inexpensive to produce, and thus they can be used as disposable devices. Also, the sample preparation will take place in a completely closed system, and thus greatly reduce the risk of infecting clinicians and/or the environment. Moreover, the samples can be prepped at the point-of-care for diagnostic procedures.

Accordingly, in another embodiment, the invention provides a method of purifying nucleic acids using the microfluidic chip of the invention.

In yet another embodiment, the invention provides a method of purifying and isolating nucleic acids using the microfluidic chip of the invention.

In yet another embodiment, the invention provides a method of purifying, isolating and detecting nucleic acids using the microfluidic chip of the invention. The detection step is preferably performed using a microarray technology attached after or at the collection reservoir of the microfluidic chip of the invention to allow detection immediately after isolation and purification, and potential amplification of the sample.

In one embodiment, the invention provides a diagnostic microfluidic chip kit for a detection of nucleic acids in a biological sample. The kit may be reusable or disposable. A one time disposable diagnostic chip kit is preferred.

The invention further provides a heterogeneous immunoassay technique for detection of pikogram (pg) levels of biomolecules. The microfluidic format makes the procedure rapid and highly sensitive. In one embodiment, one uses cyclic polyolefins as chip material. This material makes the device ideal for disposable point-of-case diagnostics.

In one embodiment, one uses immunofluorescence for detection. For even more sensitive method, such as identification of biomolecules at pikogram levels, one can use chemiluminescence.

The sensitivity of the methods of the present invention also allows use of sample materials which have low concentration of biomolecules. For example, saliva would be an ideal non-invasive biological sample material. However, its use in diagnostic methods, such as immunoassays is limited because of low concentration of biomolecules, such as cellular material in the saliva and the lack of sensitivity of the traditional methods. The sensitivity and need for only small amount of sample material in the presently disclosed system and device, makes the system ideal for diagnostic methods from saliva or other biological samples wherein concentration of a biomolecule has been too low for developing a traditional immunoassay method.

Accordingly, the invention also provides a method for isolation, purification and/or detection of biomolecules, such as proteins and nucleic acids, from biological samples such as a saliva sample.

In one embodiment, the invention provides a device and method for identification and detection of disease-causing bacteria in a point-of-care system. For example, diagnostic chip and methods for detection and diagnosis of diarrhea caused by Clostridium difficile (C. difficile) are provided.

### BRIEF DESCRIPTION OF DRAWINGS

FIGS. 1A-1B show examples of the process for manufacturing the polymeric microfluidic chip.

FIGS. 2A-2G show different examples of the views of the chip of the invention. FIGS. 2A and 2B show that the channel is filled with benzophenone and diacrylate solution and irradiated with UV light; FIG. 2C shows that a grafted polymer layer is left on the surface of the channel; and FIG. 2D shows an in situ UV-initiated polymerization to create the polymer monolith embedded with silica particles. FIG. 2E shows a schematic of the microfluidic chip of the invention. The lysis in this example was done with guanidinium thiocyanate containing buffer. The serpentine mixing channel adequately mixed the sample with the lysis agent. The isolation of nucleic acids was done with a solid-phase extraction system formed by trapping silica particles in a porous polymer monolith. After the lysate flowed over the solid-phase, wash buffer (2-propanol/water) was passed through the device to remove the proteins that adsorb onto the silica. Finally, the nucleic acids were eluted in a low stringency buffer. FIG. 2F shows a top view of a microchip with multiple channels. FIG. 2F shows a schematic representation of a lateral cross-section of an immunoassay microchannel.

FIGS. 3A-3F show example schematic illustrations of the immunoassays of the invention. In FIG. 3A, sample droplet is added on the channel inlet (i). The sample then moves through the immunoassay microchannel (ii). The “immunostack” is produced in the microchannel for chemiluminescence immunoassay—the reagents are sequentially flowed through channel from separate dispenser bottles (iii). A chemiluminescence substrate (Reagent 2) is flowed through the channel and the signal generated is captured by an on-board instant film (iv). In the example, film can be peeled from the chip and attached, for example, to a patient's history chart. Section (v) shows schematic of lateral cross section of an immunoassay microchannel that is also shown in FIG. 2G. FIGS. 3B-3E show a schematic illustration of an example of a fluorescence-based microfluidic immunoassay. FIG. 3B shows protein A immobilized on the microchannel surface and rest of the protein binding sites covered with 1% BSA. FIG. 3C shows that rabbit anti-CRP 1° Ab binds specifically to protein A. FIG. 3D shows that CRP antigen in buffer is captured by the 1° Ab. FIG. 3E shows that goat anti-CRP 2° Ab-FITC conjugate attaches to the antigen and the fluorescence is quantified. FIG. 3F shows an example of an “immunostack” produced in the microchannel for chemiluminescence-based immunoassay.

FIG. 4 shows the DNA extraction results obtained with phage λ DNA. Depicted is a comparison of the elution efficiency for consecutive DNA extractions from three different microchannels filled with the porous monolith/silica particles.

FIGS. 5A-5B show IgG immobilized on protein A (PA) versus IgG on untreated ZEONEX surface. FIG. 5A shows a fluorescent intensity profile of IgG-FITC on PA layer. FIG. 5B shows the fluorescent intensity profile of IgG-FITC on untreated surface.

FIG. 6 shows an example of fluorescence intensities for different concentrations of CRP; *p<0.05, ANOVA Single Factor.

FIG. 7 shows an example of a chemiluminescence intensity profile.

### DETAILED DESCRIPTION OF THE INVENTION

The present invention is directed to a method of manufacture of a microfluidic chip, which has channels packed with polymer-embedded particles and uses thereof. The chip of the present invention is designed for application of an untreated biological sample on the chip thus allowing isolation, purification, and detection of biomolecules, such as nucleic acids and proteins in one step. Preferably, the chip is a plastic-like material such as silicon. The invention also provides a microfluidic chip for combined isolation, purification and detection of biomolecules, such as nucleic acids and proteins thus providing a complete Lab-on-a-Chip analysis system for the biomolecules.

The microchip of the invention can be used for extraction and detection of nucleic acids from biological samples. Extraction, purification, and detection of biomolecules, such as, nucleic acids is a vital step is a number of applications, such as use of nucleic acid probes in the detection of human pathogens, food/water contaminating pathogens, plant pathogens, human or animal or plant diagnostic applications to detect polymorphisms or disease-causing polymorphisms, and pharmacogenetic applications, to detect a number of genetic markers to allow development of personalized medicine. The chip can further be used to detect nucleic acids for the purpose of identifying individuals, for criminal investigations or paternity analysis. Isolation of proteins, such as antibodies or small peptides, is also of great importance.

Thus the microfluidic chip can function as, for example, a portable disease surveillance device, a portable device to allow design of personalized medical interventions or identification of individuals. The chip can also be used for simple purification and isolation of mRNA, for example, to measure gene expression or to construct a cDNA library.

Due to its small size, the chip of the invention can also provide high-speed and high-throughput biomolecule analysis, such as nucleic acid sequence analysis.

The current commercially available biomolecule isolation systems are macroscale systems. The chip-based sample preparation system can shrink the conventional “bench-top” procedure into a miniature, portable device. Microscale system of the present invention can significantly reduce the sample and reagent consumption and allow purification of biomolecules, such as nucleic acids from small amount of any biological sample that contains a small numbers of cells. In addition, the microfluidic chips of the invention are be capable of processing different samples in parallel.

In one embodiment, the invention provides a microfluidic device comprising: (a) a substrate that is not glass with at least one channel of less than 150 μm in diameter, wherein the channel has an inlet, an outlet, and an internal space with a surface between the inlet and the outlet; (b) a first porous polymer monolith comprising a first monomer within the internal space, wherein the porous polymer monolith comprises a second monomer, and is attached to said first polymer in at least one region of the internal space, wherein the first and the second monomers may be of the same or different material; and (c) a second porous polymer monolith impregnated with particles within said internal space.

The channels of the microfluidic device of the present invention are typically about 50-300, preferably about 100-150 μm in diameter, more preferably about 100 μm in diameter. The diameter may vary depending on the desired use of the product and can be easily adjusted during the process of making of the device by the skilled artisan.

All processes, such as cell lysis, isolation of nucleic acids and recovery will be carried out on a single microfluidic chip without any sample pre-treatment. This will significantly reduce the processing time and also minimize contamination of sample. Since the microfluidic chips of the invention are made of plastic, they will be much cheaper than other microfluidic chips available in market which are made of glass or quartz. The sample preparation will take place in a completely closed environment, and thus reduce the risk of infecting the clinicians running the process.

Most currently available microfluidic devices are mad of silicon and/or glass. Use of silicon and glass is relatively expensive because of high material and manufacturing costs. Polymeric materials would be less expensive. Therefore, microfluidic devices made from polymeric materials are more suitable for mass-production of disposable devices. In one preferred embodiment, the microfluidic devices of the invention are made using cyclic polyolefin, such as ZEONEX® (ZEONEX 690R, Zeon Chemicals Inc. Louisville, Ky., USA).

For example, we determined that the mechanical and optical properties of cyclic polyolefins, such as ZEONEX are suitable for on-chip immunoassay detection.

The microfluidic device is preferably made of thermoplastic polymer that includes a channel or a multiplicity of channels whose surfaces can be modified by photografting. The device further includes a porous polymer monolith impregnated with biomolecule-binding particles, prepared via UV initiated polymerization of a porous polymer solution embedded with the particles, within the channel.

The monolith is formed by in-situ UV polymerization of a monomer mixture impregnated with for example, silica particles. For example, one can use cyclic polyolefins. In one embodiment, we used ZEONOR® or ZEONEX® (Zeon Chemicals, Louisville, Ky., USA), medical grade cyclic polyolefins, to manufacture a plastic microfluidic device. We used ZEONOR® as the primary chip material, because of its excellent mechanical properties, low autofluorescence and high UV transmission. However, any other material with suitable optical properties can be used. The optical properties necessary for both photoinitiated polymerization during manufacturing and the integration of on-chip detection in the future include good mechanical properties, low autofluorescence and high UV transmission.

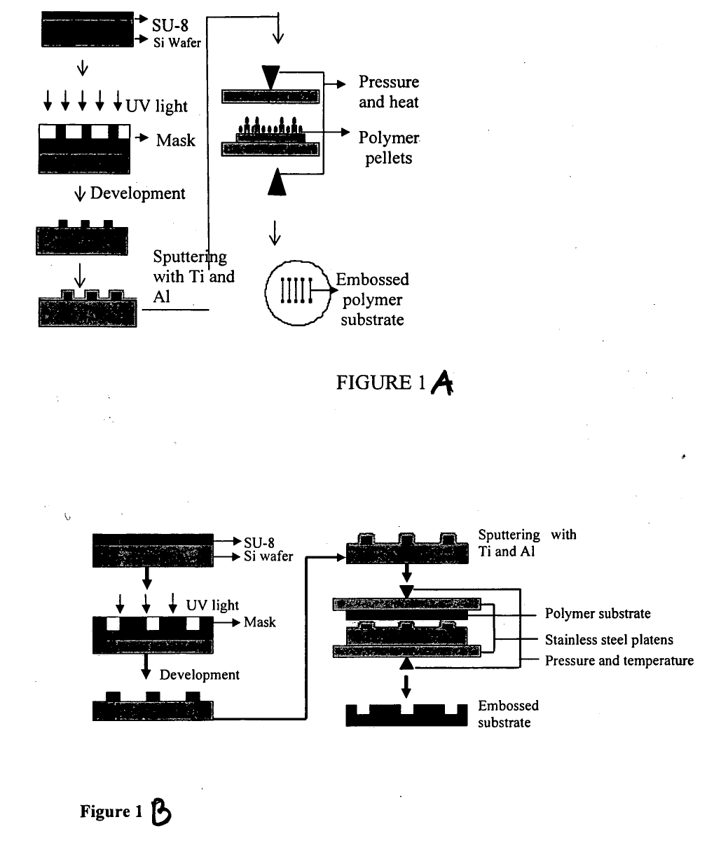

FIGS. 1A and 1B show an example process of the invention. SU-8 on Si wafer and exposed to UV light through a mask. The wafer is developed and sputtered with Ti and Al. Pressure and heat are applied and polymer pellets are added resulting in embossed polymer substrate.

In one embodiment, one can prepare the microfluidic device of the invention by hot embossing using an SU-8 master. Channels of about 100 μm and about 165 μm depths can be fabricated by this method. The width of the channels can vary from about 2 μm to at least about 500 μm. The width of the channels preferably vary from about 50 μm to about 250 μm or any width between, such as 51 μm, 52 μm, 53 μm, 54 μm, 55 μm, 60 μm, 65 μm, 70 μm, 75, 80 μm, 85 μm, 90 μm, 100 μm, 115 μm, 125 μm, 150 μm, 200 μm, or 249 μm. One can drill wells of any depth. In one preferred embodiment, one drills wells of about 1.5 mm diameter at the end of the channels for sample introduction and collection.

The SU-8 masters can be fabricated, for example, on piranha-cleaned silicon wafers by spinning SU-8 50 photoepoxy (Microchem, Newton, Mass.) or any other comparable method. In one preferred embodiment, one uses thickness of about 100 μm and about 165 μm onto the wafers.

One then pre-bakes the wafers as is known to one skilled in the art. For example, in one preferred embodiment, one pre-baked the wafers for 30 min at 95° C. After baking, the pattern is transferred through a mask preferably, by using contact lithography. Other applicable methods may be used as is known to one skilled in the art. One follows the transfer of the pattern by development, for example with SU-8 developer (Microchem) and post-baking the wafers for, for example, 1.5 h at 175° C.

In one embodiment, after the fabrication process, the SU-8 molds exhibit glass-like mechanical properties and have the negative pattern of the channels.

In one preferred embodiment, the wafers are sputter coated with about 500 Angstroms (Å) of titanium (Ti) for adhesion, followed by about 1000 Å of Al.

In one preferred embodiment, one forms the microchannels by hot embossing with a master at about 100° C. (about 30° C. above the T_{g} of ZEONOR) and about 250 psi for about minutes using, for example, a hot press, such as Heated Press 4386, Carver, Wabash, Ind. The master and the substrate can be manually separated at the de-embossing temperature, 60° C. Aluminum (Al) coating on the master facilitates easier removal of the master from the substrate after the embossing is completed. To seal the channels, another piece of ZEONOR of the same dimensions can be thermally bonded on top, for example using 68° C., 250 psi, for 2 minutes.

In one preferred embodiment, the fabricated channels are surface-modified prior to the formation of the porous monolith to improve the adhesion of the monolith to the plastic device. This can be achieved by, for example, photografting the inner surface with ethylene diacrylate (EDA) through UV-initiated reactions mediated by benzophenone. For example, one can fill the microchannels with a mixture of EDA and a hydrogen abstracting photoinitiator, such as 3% benzophenone. The chip can then be UV-irradiated for suitable time, for example, about 1-5 minutes, preferably 3 minutes. The grafting step can be carried out such that it leads to very low conversion and preferably also avoids the formation of crosslinked polymer within the channels. The excess monomer is preferably removed from the channels by rinsing. Rinsing can be performed, for example, with methanol at a flow rate of about 0.1 mL/min for 1 h.

In one preferred embodiment, one forms the monolith by polymerization of a mixture of EDMA and BuMA. The permeability of the polymer monolith typically depends on its porosity. Porogenic solvents are therefore an essential part of the polymerization mixture. The porogenic solvents dissolve all the monomers and initiator to a form a homogeneous solution and control the phase separation process during the polymerization in order to achieve the desired pore structure. For example, a porogenic mixture of 1-dodecanol and cyclohexanol has been shown to be suitable for the preparation of porous monolithic columns. In one preferred embodiment, one uses 2,2-Dimethyl-2-phenylacetophenone (DMPAP) as the UV initiator.

One can then fill the surface modified chips with the sub-micron sized silica particles, and then, preferably, a mixture consisting of BuMA (24% wt), EDMA (16% wt), 1-dodecanol (42% wt), cyclohexanol (18% wt) and DMPAP (1% wt with respect to monomers) is flowed through the channel. The microchip is then preferably irradiated with UV for about 2 minutes and washed with, for example, methanol for 12 h at a flow rate of 0.1 mL/min.

### Types of Thermoplastic Materials for Substrates

The photografting method used in preparing the microfluidic chips of the present invention can be used for the surface modification of a wide range of thermoplastic polymers. The preferred substrates, i.e. for forming channel or tube surfaces, are selected from the group consisting of poly(methyl methacrylate), poly(butyl methacrylate), poly(dimethylsiloxane), poly(ethylene terephthalate), poly(butylene terephthalate), hydrogenated polystyrene, polyolefins such as, cyclic olefin copolymer, polyethylene, polypropylene, and polyimide. Polycarbonates and polystyrenes may not be transparent enough for efficient UV transmission and therefore may not be suitable for use as substrates.

Optical properties such as light transparency at the desired wavelength range and low background fluorescence are important characteristics of substrate materials that show potential for use in the microfluidic devices of the invention. Since the photografting reactions must occur within the channels on all sides, the light must first pass through a layer of this polymer. Therefore, the substrate materials should be transparent in a wavelength range of 200 to 350 nm, preferably at any point in the range between 230-330 nm such as 250 to 300 nm, 260 to 295, etc.

In addition, the chemical properties and solubility of substrates can be taken into consideration. For instance, substrates that dissolve only in solvents, such as toluene and hexane, that are less likely to be used in standard microfluidic applications, make more desirable candidate substrate materials for photografting.

One important consideration in choosing substrate material for grafting is the grafting efficiency, expressed as N_{eff}, of the monomer to the substrate, which depends on properties such as the chemistry and transparency for light at the desired wavelength range. Grafting efficiency values of substrates correlate well with the irradiation power, the measured values of contact angles and the transparency of the substrate. An opaque substrate with a grafting efficiency value of 0 would reflect a sample, wherein no transmitted light can be detected using the material as a filter and no grafting is achieved even after 30 minutes of irradiation.

Thickness of only a few micrometers of a UV absorbing material or solution could decrease the intensity of the UV light and, consequently, the grafting efficiency. The depth of features in typical microfluidic devices may reach several tens of micrometers. Therefore, it is important to assess the effect of UV transparency of the grafting monomer mixtures during the grafting more exactly in order to determine the depth of the channel through which sufficient grafting can be safely achieved with the chosen monomer mixture. In general, the channel depth should be 10-500 μm, preferably any range between 10-250 μm including 50-250 μm, most preferably 10-50 μm. The thickness of the channel can be varied depending on the biomolecule one is looking at. For example, from 35 μm to 300 μm, and all ranges in between. Preferably from 50 μm to 250 μm. Wells are prepared to introduce and collect samples at the ends of the channels. These can range from 0.5 mm to 2.0 mm, and all ranges in between, such as 1.5 mm.

Compositions of First Monomer and its Mixtures—Mixtures Used for Photografting to the Substrate to Form a Binding Surface or a Thin Interlayer Polymer

Compositions of the grafting monomer mixtures useful for photografting are generally comprised of a bulk polyvinyl monomer, a bulk monovinyl monomer, or solutions of both a polyvinyl and monovinyl monomer, in a solvent and in the presence of 0.1 to 5% photoinitiator, preferably with 10 to 30% of monomer in the solution and 0.1 to 1% of photoinitiator, even more preferably about 10-20% monomer and 0.2-0.3% photoinitiator. For example. mixtures, such as those used in the U.S. Patent Application No. US2004/0101442 can be used.

Preferably, the thin interlayer polymer contains unreacted double bonds, which are consequently used to covalently attach the monolith containing the silica particles to the microchannel surface.

Suitable polyvinyl monomers for the first monomer for photografting the substrate include alkylene diacrylates and dimethacrylates, alkylene diacrylamides and dimethacrylamides, hydroxyalkylene diacrylates and dimethacrylates, oligoethylene glycol dimethacrylates and diacrylates, alkylene vinyl esters of polycarboxylic acids, wherein each of the aforementioned alkylene groups consists of 1-6 carbon atoms, divinyl ethers, pentaerythritol di-, tri-, or tetramethacrylates or acrylates, trimethylopropane trimethacrylates or acrylates, alkylene bis acrylamides or methacrylamides, and mixtures thereof.

Monovinyl monomers suitable for grafting the microfluidic chips of the invention include but are not limited to acrylic and methacrylic acids, acrylamides, methacrylamides and their alkyl derivatives, alkyl acrylates and methacrylates, perfluorinated alkyl acrylates and methacrylates, hydroxyalkyl acrylates and methacrylates, wherein the alkyl group consists of 1-10 carbon atoms, oligoethyleneoxide acrylates and methacrylates, acrylate and methacrylate derivatives including primary, secondary, tertiary and quarternary amine and zwitterionic functionalities, and vinylazlactones, and mixtures thereof.

In preferred embodiments, the monomers are selected for photografting a thermoplastic substrate selected from the group consisting of methyl acrylate and methacrylate, butyl acrylate and methacrylate, tert-butyl acrylate and methacrylate, 2-hydroxyethyl acrylate and methacrylate, acrylic and methacrylic acid, glycidyl acrylate and methacrylate, 3-sulfopropyl acrylate and methacrylate, pentafluorophenyl acrylate and methacrylate, 2,2,3,3,4,4,4-heptafluorobut-yl acrylate and methacrylate, 1H,1H-perfluorooctyl acrylate and methacrylate, acrylamide, methacrylamide, N-ethylacrylamide, N-isopropylacrylamide, N-[3-(dimethylamino)propyl]methacrylamide, 2-acrylamido-2-methyl-1-propanesulfonic acid, 2-acrylamidoglycolic acid, [2-(methacryloyloxy)ethyl]-trimethylammonium chloride, [2-(methacryloyloxy)ethyl]dimethyl(3-sulfopropyl)ammonium hydroxide, and 2-vinyl4,4-dimethyl-azlactone.

A variety of different chemistries can be used in microfluidic devices. The grafting conditions optimized for a number of monomers including perfluorinated, hydrophobic, hydrophilic, reactive, acidic, basic, and zwitterionic monomers, which cover a broad range of properties, can be used as described in the U.S. Patent Application No. US2004/0101442. Monomer groups in which the hydrogen abstraction readily occurs are preferred.

In some embodiments, it is preferred that the monomers for grafting exhibit a grafting efficiency of 1 or close to 1. However, since the goal is to photograft the surface with the desirable chemistry, it may be preferable to use monomers that are available despite their lower grafting efficiencies to produce the desired result.

A photomask can be attached prior to photoinitiation to permit grafting only in desired areas. However, a microfluidic chip prepared using no photomasks are preferred.

Solubility of some photoinitiators may be poor. Its higher concentration in solution can be achieved by adding a surfactant. However, while such surfactants may be used, their use is not highly recommended for grafting the first monomer to substrates. A drawback of the addition of surfactants is that mixtures may become turbid and affect grafting. Therefore, solutions containing the initiator and the surfactant should be closely monitored for clarity and transparency. Suitable surfactants include, but are not limited to, a block copolymer surfactant such as PLURONIC®, random copolymers of ethylene oxide and propylene oxide such as UCON™, and a polyoxyethylene sorbitan monooleate such as TWEEN®. All mixtures should be deoxygenated by purging prior to use in photografting.

Photoinitiator molecules for use in grafting monomers to thermoplastics are preferably aromatic ketones, including but not limited to, benzophenone, 2,2-dimethoxy-2-phenylacetophenone, dimethoxyacetophenone, xanthone, thioxanthone, their derivatives, and mixtures thereof.

In general, the extent of grafting can be controlled by irradiation time. Photoinitiated grafting should occur for all substrates to a low conversion. The irradiation time may vary but typically it is from 0.5 to 10 minutes, preferably about 2 to 5 minutes.

During photoinitiated grafting, an increase in viscosity of the monomer or its solution is observed which indicates the concomitant formation of a considerable amount of polymer in the solution. The extent of this polymerization can be reduced by diluting the monomer with a suitable solvent. Suitable solvents should be capable of solubilizing the grafted monomer. Dilution with a solvent that has lower absorbency in the UV range than the monomer itself also helps to reduce the negative self-screening effect of the monomer. Examples of suitable solvents include water, alcohols, such as tert-butyl alcohol (tBuOH), and their mixtures.

A very short, such as about 3 minutes., irradiation and reaction time is preferred to avoid the rapid crosslinking if a pure divinyl monomer is used for photografting. However, if the reaction time is not sufficient to achieve the desired extent of surface modification, the grafting time can be extended or the monomer mixture can be changed, for example, by using a 1:1 mixture of divinyl and monovinyl monomer. A monovinyl monomer used in the grafting monomer solution decreases the crosslinking density of the grafted surface layer enabling it to swell in the polymerization mixture used later for the preparation of the monolith.

### Preparation of Porous Polymer Monoliths Through Photopolymerization of Second Monomer Mixture

A porous polymer monolith useful for the preferred embodiment is a solid polymer body containing a sufficient amount of pores of sufficient size that enable convective flow. Preferred monoliths are those as disclosed in U.S. Pat. Nos. 5,334,310; 5,453,185; and 5,929,214, the subject matters of which are hereby incorporated by reference for purposes of describing monoliths. The preferred polymer monolith is prepared by polymerizing a polyvinyl monomer or, more preferably, a mixture of a polyvinyl and monovinyl monomer, in the presence of an initiator, and a porogen. The polymerization mixture is added to the channel and polymerization is initiated by UV irradiation therein so as to form the polymer monolith. The polymer monolith is then washed with a suitable liquid to remove the porogen.

In a preferred embodiment, the polymerization mixture is comprised of about 24 wt % monovinyl monomer, about 16 wt % polyvinyl monomer, and about 60 wt % porogens, whereby the photopolymerizations are carried out at room temperature. The ranges of each of the monomer, crosslinker and porogens can be varied according to the methods described in U.S. Pat. Nos. 5,334,310; 5,453,185; and 5,929,214.

The polyvinyl monomer is generally present in the polymerization mixture in an amount of from about 10 to 60 wt %, and more preferably in an amount of from about 20 to 40 wt %. Suitable polyvinyl monomers include alkylene diacrylates and dimethacrylates, hydroxyalkylene diacrylates and dimethacrylates, alkylene bisacrylamides and bismethacrylamides, wherein the alkylene group consists of 1-6 carbon atoms, oligoethylene glycol diacrylates and dimethacrylates, diallyl esters of polycarboxylic acids, divinyl ethers, pentaerythritol di-, tri-, or tetraacrylates and methacrylates, trimethylopropane triacrylates and trimethacrylates, and mixtures thereof.

Preferred monovinyl monomers include but are not limited to, acrylic and methacrylic acids, acrylamides, methacrylamides and their alkyl derivatives, alkyl acrylates and methacrylates, perfluorinated alkyl acrylates and methacrylates, hydroxyalkyl acrylates and methacrylates, wherein the alkyl group consists of 1-10 carbon atoms, oligoethyleneoxide acrylates and methacrylates, vinylazlactones, acrylate and methacrylate derivatives including primary, secondary, tertiary, and quarternary amine functionalities and zwitterionic functionalities, and mixtures thereof.

The porogen used to prepare the monolith may be selected from a variety of different types of materials. For example, suitable liquid porogens include aliphatic hydrocarbons, esters, alcohols, ketones, ethers, solutions of soluble polymers, and mixtures thereof. The porogen is generally present in the polymerization mixture in an amount of from about 40 to 90 wt %, more preferably from about 60 to 80 wt %.

In a preferred embodiment, the composition of porogenic solvent is used to control porous properties. The percentage of decanol in the porogenic solvent mixture with a co-porogen, such as cyclohexanol or butanediol, affects both pore size and pore volume of the resulting monoliths. A broad range of pore sizes can easily be achieved by simple adjustments in the composition of porogenic solvent.

In contrast to the pore size, the type of porogen has only a little effect on the pore volume since, at the end of the polymerization, the fraction of pores within the final porous polymer is close to the volume fraction of the porogenic solvent in the initial polymerization mixture because the porogen remains trapped in the voids of the monolith.

In one preferred embodiment, the pore size would depend on the ultimate use of the porous polymer monolith. A preferred pore size in a preferred embodiment is greater than about 600 nm because this size enables flow through at a useful velocity and reasonable back pressure. However, smaller pores also may be useful and suitable.

Efficient polymerization of the porous polymer monolith is achieved by using free radical photoinitiators. In the preferred embodiment, about 0.1 to 5 wt % with respect to the monomers of hydrogen abstracting photoinitiator can be used to create the porous polymer monolith. Typically, 1 wt % with respect to monomers of a hydrogen abstracting photoinitiator including, but not limited to, benzophenone, 2,2-dimethoxy-2-phenylacetophenone, dimethoxyacetophenone, xanthone, thioxanthone, their derivatives and mixtures thereof is used.

Surfactants, such as PLURONIC F-68, can be added to improve the solubility of photoinitiators. Suitable surfactants include, but are not limited to, a block copolymer surfactant such as PLURONIC®, random copolymers of ethylene oxide and propylene oxide such as UCON™, and a polyoxyethylene sorbitan monooleate such as TWEEN®. All mixtures should be deoxygenated by purging prior to use in photografting.

### Polymerization of the Channel-Filling Porous Polymer with Particles

The solid phase of the microfluidic chip of the invention is made by in-situ UV polymerization of the monolith column impregnated by particles, such as silica particles.

Suitable nucleic particles include silica particles, silica-particles with different functional groups, such as as-NH₂, and —COOH (Kisker Biotech), magnetic silica particles, such as MAGPREP® Silica Particles (Merck, Darmstadt, Germany), and the like.

After the porous polymer monolith has been polymerized and prepared in the channel or capillary, the channel is filled with the functional monomer impregnated with particles, preferably silica particles. A mixture of more than one monomer, or their solution can also be used. The polymer-particle-filled channels are then irradiated. Alternatively, the monomer mixture may further comprise a solvent.

The monomer mixture is deaerated and then pumped to fill the pores of the monolith. The mixture is generally comprised of a bulk monomer or its 10 to 50% solution in a solvent and 0.1 to 5% photoinitiator, preferably I 0 to 30% of monomer in the solution and 0.1 to 1% of photoinitiator.

Grafting is preferably achieved by UV irradiation of a stationary porous monolith filled with the monomer/particle solution through a mask from a sufficient distance for a sufficient period of time to -raft polymer chains having functional groups to the monolith. When the irradiation step is complete, the capillary is then washed to remove residual monomer solution. Any solvent that dissolves the residual polymer can be used to wash the capillary.

Suitable monomers for photografting porous polymer monoliths impregnated with particles, possess a variety of functionalities, but are in no way limited to, hydrophilic, hydrophobic, ionizable, and reactive functionalities.

Examples of suitable monomers for photografting porous polymer monoliths include, but are not limited to, methyl acrylate and methacrylate, butyl acrylate and methacrylate, tert-butyl acrylate and methacrylate, 2-hydroxyethyl acrylate and methacrylate, acrylic and methacrylic acid, glycidyl acrylate and methacrylate, 3-sulfopropyl acrylate and methacrylate, pentafluorophenyl acrylate and methacrylate, 2,2,3,3,4,4,4,4-heptafluorobutyl acrylate and methacrylate, 1H,1H-perfluorooctyl acrylate and methacrylate, acrylamide, methacrylamide, N-ethylacrylamide, N-isopropylacrylamide, N-[3-(dimethylamino)propyl]methacrylamide, 2-acrylamido-2-methyl-1-propan-esulfonic acid, 2-acrylamidoglycolic acid, [2-(methacryloyloxy)ethyl]-trim-ethylammonium chloride, [2-(methacryloyloxy)ethyl]dimethyl(3-sulfopropyl)a-mmonium hydroxide, and 2-vinyl-4,4-dimethyl-azlactone.

Solubility of some photoinitiators may be poor. Its higher concentration in solution can be achieved by adding a surfactant. However, use of surfactants is not highly recommended. A drawback of the addition of surfactants is that mixtures may become turbid, and thus not allow irradiation with UV light and affect grafting. Therefore, solutions containing the initiator and the surfactant should be closely monitored for clarity and transparency.

In a preferred embodiment, the desirable solvent for use in photografting polymer monoliths (i) should not absorb excessively in the UV range to exert minimum self-screening effect, (ii) should not allow hydrogen abstraction, thereby being incorporated into the polymer layer by termination reactions and/or initiate undesired homopolymerization, and (iii) must dissolve all components of the third monomer mixture (monomer and initiator). A preferred solvent is water, t-butanol (tBuOH) and its mixtures with water, that all meet these criteria.

The preferred embodiment enables the functionalization by photoinitiated grafting of porous materials located within capillaries, microfluidic channels, and other suitable devices. Functionalization permits porous polymer monoliths within the capillaries and channels of microfluidic and other devices to be used for various procedures such as mixing, concentrating, and separation reactions. Thus, the preferred embodiment facilitates the design and preparation of numerous functional elements that are instrumental to the development of complex microanalytical elements and systems.

Furthermore, a major advantage of the microfluidic chips and methods described herein is the ability to pattern grafted areas thus facilitating preparation of materials with different spatially segregated chemistries within a single porous polymer monolith with nucleic acid-binding particles. Functionalization of several areas can be controlled in terms of placement and extent as simultaneous or sequential functionalizations are possible.

The additional benefit of photoinitated grafting is the ability to create patterns differing in properties such as surface coverage or type of the grafted chemistry. By placing masks over certain areas of the porous polymer monolith, patterns of different functionalities can be created. The sharp edges of the patterned features enable placing different functionalities within a porous polymer monolith next to each other with no dead volume between the functionalities, thereby allowing different elements to be placed directly adjacent to each other. In contrast to the typical “homogenous” grafting, the preparation of monoliths with longitudinal gradients of surface coverage or combining different chemistries using masks with a gradient of transparency for UV light is also contemplated by the invention.

Photografting also facilitates the preparation of layers of functionalities in a porous polymer monolith in both axial and radial direction with respect to the direction of flow.

The qualitative effect of the intensity of the UV light on the grafting efficiency is different polymers can be used as filters to modulate intensity. The use of a photomask, such as a multi density resolution mask (Series I, Ditric Optics, Hudson, Mass.), that includes several fields differing in UV light transmittance enables creation of creation of gradients. Grafting through masks with a gradient of absorbency enables the fabrication of layers with both stepwise and continuous gradients of hydrophilicity, polarity, acidity, or combinations thereof, along the channel by simply using multidensity, continuous gray-scale photomasks, a moving shutter or the like.

One of the reasons for the photografting surfaces of thermoplastic substrates is to modify the walls of channels in microfluidic devices to hold porous polymer monoliths. Any known photografting methods can be used. The channel walls in a microfluidic chip are preferably photografted as described in the U.S. Patent Application No. US2004/0101442 to achieve a firm covalent bond between the channel wall and porous polymer monoliths. This method described herein prevents the formation of voids at the monolith-wall interface.

The chip was prepared by hot embossing with an SU-8 master. Prior work in hot embossing microscale features into polymeric substrates used nickel alloy molds made with L]GA or electroforming, which can be very cost intensive. Our rapid prototyping process involves embossing directly from the SU-8 master. The chips fabricated by the hot embossing process were then used for on-chip isolation of nucleic acids. The chip is made by hot embossing with a mold under high temperature and pressure. The mold itself can be made by LIGA, metal electroform made by electroplating, etching glass or silicon, epoxy based photoresists such as SU-8, and CNC milling of a metal piece. In one preferred embodiment, one uses SU-8 molds and etched silicon molds, since they are the most inexpensive techniques. For large production of the device, other methods such as metal electroform or LIGA is more applicable.

The device can also be made by injection molding of the same polymer material.

Attaching the solid phase to the walls of the micro channels—Due to the relatively inert properties of the polymeric channel surfaces, it is difficult to achieve good bonding of the solid phase with the native walls of the plastic devices. Silane primer reagents, such as 3-(trimethoxysilyl)propyl methacrylate, can be easily used to functionalize the walls of the channels made in glass or silicon. However, no such surface primers are readily available for pretreatment of polymer surfaces, so other surface modification methods, such as polymer grafting have to be applied. In our case, the crafting was done via photoinitiated polymerization prior to the formation of the monolith. The grafted interlayer polymer covalently attaches to the monolith and prevents the formation of voids between the monolith and the channel surface. The interlayer also stops the monolith from migrating down the channel during separations. The high UV transmission of ZEONOR makes it suitable for in-situ photopolymerization applications. Photopolymerization of monolith embedded with silica particles is an easy alternative to the widely-used silica bead/sol-gel approach. Stachowiak et al. demonstrated the formation of polymer monolith inside of a cyclic olefin polymer.

Material selection: Any engineering polymer that satisfies the following criteria can be used to make the device. The polymer should be compression moldable, it should not be excessively autofluorescent, and it should be transparent to UV light for easy curing of the solid phase and transparent at 488 nm and 530 nm for conventional detection methods

There are several commercial engineering polymers that meet these criteria such as polymethyl methacrylate (PMMA), polycarbonate (PC), and several proprietary cyclic olefin materials (such as ZEONOR and ZEONEX). Cyclododecatriene A high-purity, liquid cyclic polyolefin, DuPont; Cyclododectriene (CDDT), a high purity, liquid cyclic Polyolefin, CAS Number: 4904-61-4; (poly(methyl methacrylate)), or cyclic polyolefin; cyclic polyolefin polymer (ZEONEX), ZEON corporation.

### USES OF THE MICROFLUIDIC DEVICE OF THE INVENTION

### Isolation of nucleic acids

We have developed a method of separating nucleic acids from crude cell lysates using the disposable plastic microfluidic device of the invention. The solid phase extraction columns of the microfluidic device of the invention are capable of binding, concentrating and eluting nucleic acids from mammalian cell and lysate samples of about 100 microliters or less. This technological development, when combined with parallel progress in chip-based polymerase chain reaction and fluorescence detection provides a superior differential diagnosis of infections at the point of care.

Solid phase extraction (SPE) is an important and widely used sample preparation technique, which allows both the purification and preconcentration of biological samples. The purification of nucleic acids is usually done with solid-phase extraction on silica resins. Extraction is achieved because nucleic acids have the tendency to bind to silica in the presence of a high concentration of chaotropic salt. The extracted nucleic acids are subsequently eluted in an aqueous low-salt buffer and concentrated into a very small volume. The time necessary for nucleic acid purification w,as greatly reduced when the original phenol extraction method was replaced by silica based solid-phase extraction systems. SPE methods for DNA extraction have since been successfully miniaturized and incorporated in microfluidic chips. The sol-gel/silica bead mixtures have been shown to have very good extraction efficiencies and reproducibility in microfluidic systems. However, the sol-gel process involves high temperatures and is not suitable for use in polymeric devices.

The method of immobilizing silica particles in a porous polymer monolith to form a microscale solid-phase extraction system is described, supra. Monolithic materials have been successfully used in a wide variety of applications, including capillary electrochromatography, micro-mixers and electroosmotic pumps. The monolithic column was formed by in situ UV polymerization of a monomer mixture impregnated with silica particles. The solid-phase was covalently attached to the walls of the microchannels to prevent its displacement when samples were flowed through the channels. We have demonstrated the ability of these monoliths to extract DNA from simulated sample solutions.

Microfluidic approaches to DNA purification have been previously demonstrated in glass microchips fabricated by Deep Reactive Ion Etching (DRIE). Recovery of DNA molecules was achieved by packing microchannels with silica particles and immobilizing by a sol-gel method. By heating a slurry of tetraethylortho-silicate (TEOS), ethanol and silica particles, a monolith that is covalently attached to the walls of the glass microchip is achieved. Replicating the sol-gel chemistry in a plastic chip is difficult, since the process involves temperatures higher than the T_{g} (glass transition temperature) of most engineering polymers. Also in case of polymer microchannels, a major challenge is getting the monolith to adhere to the walls.

In this work, we stepped aside from the traditional sol-gel approach, and have used a porous polymer monolith to embed silica particles. Photopolymerization of monolith embedded with silica particles is an easy alternative to the widely-used silica bead/sol-gel approach.

Stachowiak et al. (Electrophoresis 24, 3689-93, 2003) demonstrated the formation of a polymer monolith within a cyclic olefin polymer. However, the use of the polymer monolith to entrap silica particles has not been previously shown. The channel walls are modified by a polymer photografting method lo encourage formation of covalent bonds with the monolith. The technique allows successful extraction and elution of nucleic acids.

Existing DNA isolation techniques typically lyse cells outside the microchip with conventional methods before the on-chip experiment, and microliters of the cell lysate or purified DNA sample were loaded onto the chip for DNA isolation the present invention differs from the existing methods. Cells lysis is done on the chip without the need for pretreating the sample. Typically, only samples that are not fluid enough to be applied through the inlet of the channel of the chip may need to be mixed with a buffer before application of the sample into the channel. For example, one can use chaotropic agents and do purification of nucleic acids on the same chip without any sample pretreatment.

Isolation of desired nucleic acids. We can successfully isolate nucleic acids from mammalian cells. Bacterial cell walls are much more robust and often require more vigorous lysing steps. The presence of the more robust bacterial cell walls also acts to plug an SPE column that has pores that are too small. Accordingly, we fabricated and tested a range of columns using different amounts of porogen. Typical isolation procedure comprises the following steps: 1) Obtain a biological sample; 2) Optionally, culture the sample at appropriate temperature, for example at 37° C., in an appropriate culture medium; 3) Optionally, chemically lyse bacteria in an appropriate buffer system; 4) Run bacterial sample over micro-SPE columns; 5) Wash column; 6) Extract isolated nucleic acids; 7) Remove isolated and concentrated nucleic acids from chips; 8) Run polymerase chain reactions using primers designed to detect a nucleic acids present in the bacteria to be detected.

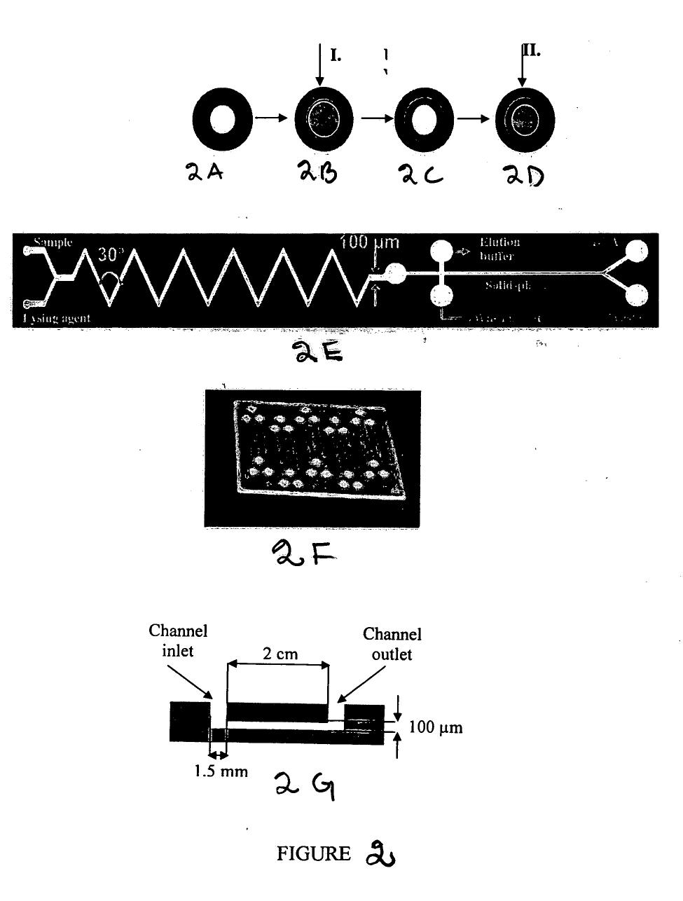

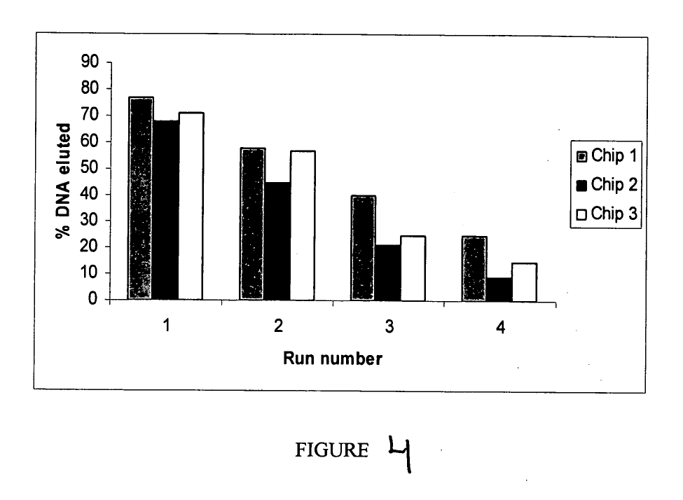

For example, FIG. 2E shows a schematic of a preferred embodiment of the chip or device of the invention. One can lyse the biological sample including eukaryotic and prokaryotic cells, with, for example, guanidinium thiocyanate containing buffer. The mixing channel is preferably in the form of a serpentine mixing channel that can adequately mix the sample with the lysis agent. It is specifically noted that the invention is not limited to any particular shape of the device or the channels. The skilled artisan can readily alter the geometries of the device based upon the present description and examples. Accordingly, any suitable geometric format can be used according to the teachings of the present invention. The isolation of nucleic acids can be done with a solid-phase extraction system formed by trapping silica particles in a porous polymer monolith. After the lysate flows over the solid-phase, wash buffer (2-propanol/water) will be passed through the device to remove the proteins that adsorb onto the silica. Finally, the nucleic acids will be eluted in a low stringency buffer. FIG. 4 shows an example of nucleic acid recovery using the present invention.

### Immunoassays

On can use the microfluidic device as described above to isolate, purify and detect biological molecules. For example, one can use the microfluidic device to do highly sensitive and effective immunoassays.

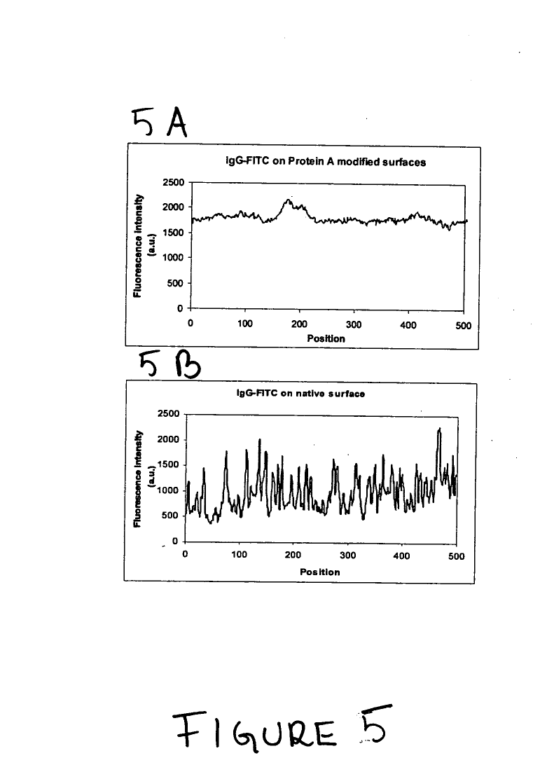

To increase the sensitivity of the immunoassay using the device of the invention, one preferably immobilizes a Protein A (PA) layer on the surface of the channels. PA has four high affinity binding sites for immunoglobulin G (IgG) of most species. Accordingly, binding to PA allows correct alignment of antibodies to receive the antigens one wishes to detect in the sample. FIG. 5 illustrates the improved signal quality when one uses PA-modified surface. In this example, 0.1 mg/mL of PA can be physisorbed on the surface of the channel and 10 μg/mL of rabbit IgG-FITC conjugate was added to the PA layer (FIG. 5A) and native surface (FIG. 5B).

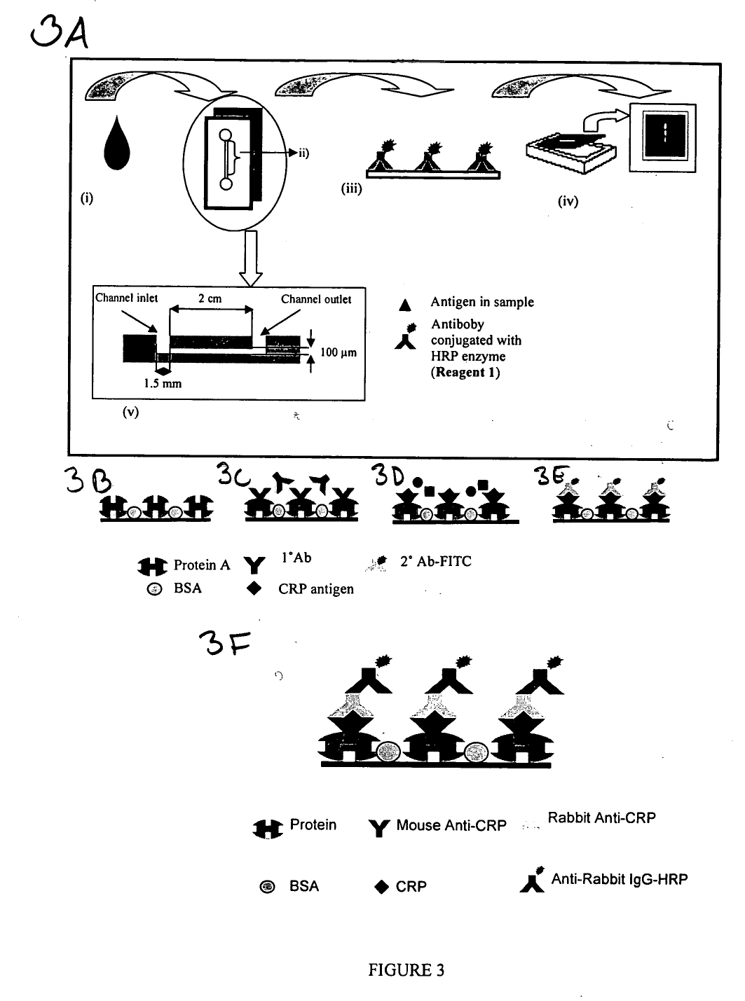

Immunoassays can be performed using, for example, radioactive detection systems, immunofluorescence or chemiluminescence. Preferably, immunofluorescence or chemiluminescence is used. For analysis of a sample that is suspected to contain small amounts, such as pikogram quantities of the biomolecules one wishes to detect, one preferably uses chemiluminescence-based methods. FIG. 3 illustrates a non-limiting example of immunoassay configurations that one can use according to the present invention.

The fluorescence detection method can be based, for example, on a heterogeneous sandwich assay. In sandwich immunoassays, a monoclonal antibody specific to the target analyte (antigen), is bound to a surface. The sample fluid is contacted with the surface, whereby the antibody captures the target antigen. A labeled polyclonal antibody attaches to the antigen to complete a “sandwich”. The label, for example, a linked enzyme, a fluorophore, or a radionuclide, generates a signal that is detected to quantify the captured antigen. Sandwich immunoassay is the most sensitive and specific immunoassay technique for antigen detection. However, it is not desirable in a conventional immunoassay setup, because it involves many fluid handling steps for sample/reagent loading and washing. A microfluidic immunoassay method easily overcomes this drawback, because the reactions are controlled by simply pumping solutions into the channels of the chip sequentially.

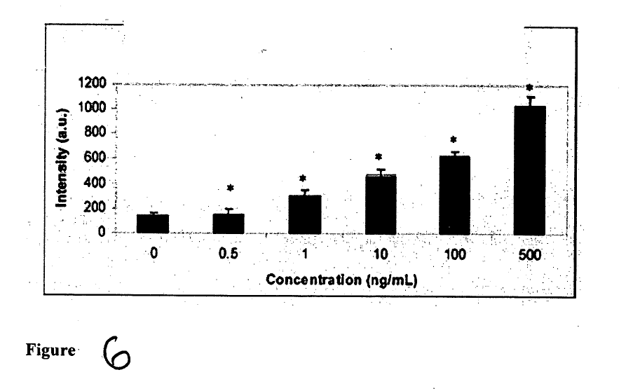

FIG. 6 illustrates one test result of detection limits using fluorescence detection in the immunoassay of the invention.

Chemiluminescence is a highly sensitive technique with limits of detection in the low pikogram range. Chemiluminescence based immunoassay can be performed with, for example, luminol as the substrate and horseradish peroxidase (HRP) as the enzyme conjugated to the secondary antibody. In the presence of hydrogen peroxide, HRP catalyzes the oxidation of luminol. When oxidized luminol returns to its original state, an iridescent blue light is emitted, which can be detected by exposure to X-ray film, instant film, or an imager capable of detecting chemiluminescent signals. We used VERSADOC™ imaging system from Bio-Rad Laboratories, Inc. (Hercules, Calif.) to detect the chemiluminescent signals. Other chemiluminescence systems are well known to one skilled in the art, and can equally well be used in the methods of the present invention.

An example schematic of a chemiluminescent immunoassay is shown in FIG. 3F. The channel surfaces were modified with protein A and 1% BSA as mentioned in the immunofluorescence technique. The steps of the immunoassay are given in Table 1, where the primary and secondary antibodies are denoted as 1* Ab and 2° Ab respectively. After each incubation step, the channels are washed. Washing can be performed, for example, using 1× PBS by aspiration. Other buffers can be used as is known to one skilled in the art. The chemiluminescent signals can be measured, for example about 2-3 min after the substrate is loaded into the channel.

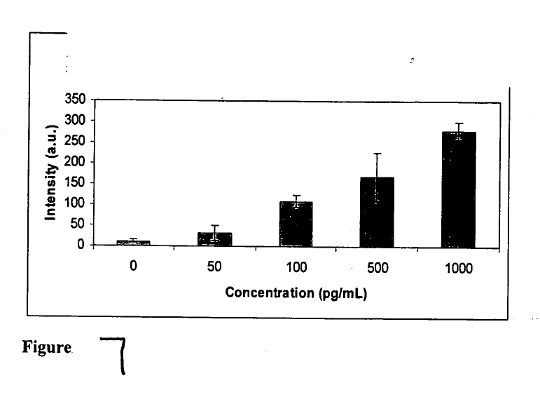

FIG. 7 shows that using chemiluminescence detection even higher sensitivity can be achieved.

### Detection of Biomolecules in a Biological Sample

Since 1983, PCR has allowed not only for the detection of an infectious agent but also for its identification through the amplification of specific molecular markers. The advent of microfluidic technology in the early 1990's held the promise of easy to use, minimally invasive, point-of-care diagnostic devices that exploit molecular techniques. In fact, many biochemical methods including separations of proteins, nucleic acids, and performance of PCR have been miniaturized in the research lab as successful proofs of concept. By obviating the need for a full diagnostic laboratory, advanced, specialized laboratory tests once thought impractical or too costly to perform in remote areas, field hospitals, and small clinics will become routine.

Simple, inexpensive diagnostics will have an impact in several broad areas of general interest, such as homeland security, differential diagnosis in nursing homes and hospitals, in remote low income areas and the developing world. Many agents considered likely for use in a biological attack against military or civilians present with common symptoms in the clinic. Only after close observation of the first few “beacon” cases will clinicians be able to conclusively diagnose the presence and nature of a biological attack. The time lost in making these distinctions using traditional diagnostic techniques that require a full scale laboratory and skilled labor will likely lead to spread of an outbreak before containment procedures can be initiated. In addition, many antibiotic treatments are most effective if they are initiated before the onset of major symptoms.

Common difficult-to-diagnose infections are responsible for hundreds of thousands of deaths in the U.S. each year. For example, based solely on the symptoms, it is virtually impossible to know whether a diarrheal illness will have a progressive and/or fulminant course. Thus, the availability of a simple, rapid, low-cost, sensitive and specific diagnostic test would permit the delivery of directed treatment for many acute diarrheas. A case in point is colitis due to Clostridium difficile. C. difficile is the most common cause of diarrhea spread in hospitals and nursing homes in the United States and is increasingly a major cause of morbidity and mortality among the elderly in acute and chronic healthcare facilities. Ideally, additional antimicrobial therapy should be initiated early, but no sensitive, specific, and reliable test exists for making a diagnosis of C. difficile associated diarrhea at the initial point of care. Current testing, including cytotoxicity and immunoassays require hours to days to complete, a time frame where treatment delay could extend disease complications. Even small improvements in the speed of diagnosis of treatable infectious disease could have major impacts on all hospital and nursing home populations but would be especially important in low-income or remote areas. We used C. difficile as a model organism (a non-infectious strain) to test our device. Naturally, our results are applicable for diagnosis of any bacterial, viral, or parasite presence in a biological sample.

The biological sample as used in the present invention can be any material that either contains or is suspected to contain the biomolecules, such as nucleic acids or proteins that one desires to detect, extract or purify. The sample may be blood, serum, sputum, saliva, urine, stool, bone marrow, consumable food/drink stuff, soil, water, or any other material that can be either directly added to the channel of the microfluidic device of the invention or mixed with a small amount of buffer reagent to make the sample liquid enough to enter the channel.

The device of the present invention can be adapted to diagnose one or more, preferably multiple disease causing agents. For example, the microfluidic platform of the present invention allows one to create rapid, disposable, and inexpensive testing system for multiple infectious diseases.

We have fabricated microfluidic devices as described, supra, that lyse mammalian cells and isolate and concentrate their nucleic acids. Our rapid method is completely scalable and our microfabrication design is applicable to materials and processes used in mass production. Lysing bacterial cells in the microfluidic platform has posed a challenge in the art. While mammalian cells can be lysed by a combination of lysis buffer and simple mixing, lysing bacteria cells takes significantly more effort due to the nature of the cell wall. We show that mechanical shear induced by flow disruption in addition to mixing with a lysis buffer can break apart bacteria, such as C. difficile.

The modified microfluidic mixing channels described here, and shown, e.g. in FIGS. 2E and 2F, are sample preparation devices for lysis and extraction of nucleic acids from patient samples at the point of care. Extraction/purification of nucleic acids is an important step in molecular diagnostics that use nucleic acid probes in the detection of human pathogens. When combined with a real time PCR step, this technology enables faster, more specific detection of microorganisms in patient samples. Sample and reagent consumption will be greatly reduced. All processes will be carried out on a single chip with little sample pretreatment, significantly reducing processing time and minimizing the potential for cross contamination. The plastic chips are easily prototyped for rapid testing of new layouts. The devices are inexpensive and disposable.

Solid phase extraction (SPE) allows both the purification and preconcentration of biological samples (Weeks, B. L., et al., Scanning, 2003. 25(6): p. 297-9). The purification of nucleic acids is usually done on silica resins (Breadmore, M. C., et al., Electrophoresis, 2002. 23(20): p. 3487-95). Extraction is achieved because nucleic acids will bind to silica in the presence of a high concentration of chaotropic salt. The extracted nucleic acids are subsequently eluted in an aqueous low-salt buffer and concentrated into a very small volume. SPE methods for DNA extraction have been successfully miniaturized and incorporated in microfluidic chips. The sol-gel/silica bead mixtures have good extraction efficiencies and reproducibility in microfluidic systems (Breadmore, M. C., et al., Towards a microchip-based chromatographic platform. Part 1: Evaluation of sol-gel phases for capillary electrochromatography. Electrophoresis, 2002. 23(20): p. 3487-95; Breadmore, M. C., et al., Anal Chem, 2003. 75(8): p. 1880-6). However, the sol-gel process involves high temperatures and is not suitable for use in polymeric devices.

Our method permits immobilizing silica particles in a porous polymer monolith to form a microscale solid-phase extraction system. The monolithic column is formed by in situ UV polymerization of a monomer mixture impregnated with silica particles. The solid-phase is covalently attached to the walls of the microchannels to prevent its displacement when samples are flowed through the channels. We have demonstrated the ability of these monoliths to extract DNA from simulated sample solutions and mammalian cell lysates.

In addition to shear force, one may also use mechanical obstacles to help lyse the cells, particularly the bacterial cells. We have been successful creating composite monoliths with microscale silica particles, and can make a porous monolith impregnated with carbon nanotubes for cell lysis.

Nanotubes, as used herein, refer to typically carbon nanotubes of about 1-50 microns, preferably about 1-20 microns, or 1-10 microns long and about 10-300 nm in diameter, preferably about 30-150 nm, alternatively about 50-150 nm in diameter.

One can prepare the monolith as shown above, and in addition, substitute some or all of the silica beads with nanotubes. The resulting open pore structure thus contains exposed nanotubes. Nanotubes embedded in polymer filters have been used industrially to purify water via bacterial lysis (Srivastava, A., et al., Carbon nanotube filters. Nat Mater, 2004. 3(9): p. 610-4; Valcarcel, M., et al., Present and future applications of carbon nanotubes to analytical science. Anal Bioanal Chem, 2005. 382(8): p. 1783-90). Such nanotubes can easily be used to impregnate the internal space of at least a part of a channel of the microfluidic device of the present invention.

All the references cited throughout the specification and examples are herein incorporated by reference in their entirety.

### EXAMPLES

### Example 1

Device fabrication: The microfluidic channels were fabricated by hot embossing with an SU-8 master. Channels of 100 μm and 165 μm depths were fabricated by this method. The widths of the channels varied from 50 μm to 250 μm and wells of 1.5 mm diameter were drilled at the end of the channels for sample introduction and collection. The SU-8 masters were fabricated on piranha-cleaned silicon wafers by spinning SU-8 50 photoepoxy (Microchem, Newton, Mass.) at a thickness of 100 μm and 165 μm onto the wafers. After pre-baking the wafers for 30 min at 95° C., the pattern was transferred through a mask by contact lithography. This was followed by development with SU-8 developer (Microchem) and post-baking the wafers for 1.5 h at 175° C. After this fabrication process, the SU-8 molds exhibited glass-like mechanical properties and had the negative pattern of the channels. The wafers were then sputter coated with 500 Å of Ti for adhesion, followed by 1000 Å of Al.

The microchannels were formed by hot embossing with the master at 100° C. (30° C. above the T_{g} of ZEONOR) and 250 psi for 5 minutes using a hot press (Heated Press 4386, Carver, Wabash, Ind.). The master and the substrate were manually separated at the de-embossing temperature, 60° C. (FIGS. 1A and 1B). The Al coating on the master facilitates easier removal of the master from the substrate after the embossing is completed. To seal the channels, another piece of ZEONOR of the same dimensions was thermally bonded (68° C., 250 psi, 2 min.) on top.

Preparation of Solid-Phase: The fabricated channels had to be surface modified prior to the formation of the porous monolith to improve the adhesion of the monolith to the plastic device. This was achieved by photografting the inner surface with ethylene diacrylate through UV-initiated reactions mediated by benzophenone. The microchannels were filled with a mixture of EDA and 3% benzophenone, which is a hydrogen abstracting photoinitiator. The chip was then UV-irradiated for 3 minutes. The grafting step was carried out such that it led to very low conversion and avoided the formation of crosslinked polymer within the channels. The excess monomer was removed from the channels by rinsing with methanol at a flow rate of 0.1 mL/min for 1 h.

The monolith was formed from by polymerization of a mixture of EDMA and BuMA. The permeability of the polymer monolith depends on its porosity. Porogenic solvents are therefore an essential part of the polymerization mixture. The porogenic solvents dissolve all the monomers and initiator to a form a homogeneous solution and control the phase separation process during the polymerization in order to achieve the desired pore structure. A porogenic mixture of 1-dodecanol and cyclohexanol has been shown to be suitable for the preparation of porous monolithic columns. DMPAP w,as chosen as the UV initiator since it causes very fast polymerization, with complete conversion achieved within 10 min even at the lowest radiation power.

The surface modified chips were filled with the sub-micron sized silica particles, and then a mixture consisting of BuMA (24% wt), EDMA (16% wt), 1-dodecanol (42% wt), cyclohexanol (18% wt) and DMPAP (1% wt with respect to monomers) was flowed through the channel. The microchip was then irradiated with UV for 2 minutes and then washed with methanol for 12 h at a flow rate of 0.1 mL/min.

FIG. 3 shows the schematic ofthe chip. The lysis will be done with guanidinium thiocyanate containing buffer. The serpentine mixing channel will adequately mix the sample with the lysis agent. The isolation of nucleic acids will be done with a solid-phase extraction system formed by trapping silica particles in a porous polymer monolith. After the lysate flows over the solid-phase, wash buffer (2-propanol/water) will be passed through the device to remove the proteins that adsorb onto the silica. Finally, the nucleic acids will be eluted in a low stringency buffer.

At present, we have been successful in making the plastic chip in ZEONOR by a simple hot embossing method and incorporation of the solid-phase in the microchannels for extraction of DNA. The polymer monolith is formed by in situ UV polymerization of a monomer mixture impregnated with the silica particles. The high UV transmission of ZEONOR makes it suitable for in-situ photopolymerization applications. A photografted interlayer polymer is used to attach the monolith to the inner walls of the channel. To test the efficiency of the porous monolith in DNA extraction, we used spectroscopic measurement of absorption at 260 nm and fluorescence intensity measurement with Hoechst 33258 DNA stain. FIG. 4 shows examples of DNA extraction results obtained with phage λ DNA.

In summary, we believe that the techniques described here are a first step toward adapting the monolith technology developed in silica and glass for applications in plastic microfluidic chips. The high glass transition temperature and UV transmission of the cyclic polyolefin used in this work makes it ideal for integration of cell lysis, sample purification and amplification/ detection modules on one disposable device.

### Example 2

### Immunoassay Methods Using the Microfluidic Device of the Invention

This example describes the development of fluorescence and chemiluminescence based microfluidic immunoassay techniques on a thermoplastic microchip. The immunoassays were applied to determine femtomolar concentrations of C-reactive protein (CRP), a classic inflammation marker associated with cardiovascular diseases. Because of the very high sensitivity of the described immunoassay techniques, they are suitable for developing saliva-based diagnostic tests. The microfluidic chips were fabricated in cyclic polyolefin by hot-embossing techniques. The surface of the microchannels were modified by immobilizing protein A to increase the sensitivity of the immunoassays, since protein A has high affinity for immunoglobulin G (IgG) of most species. Concentrations of CRP were determined on-chip by both fluorescence and chemiluminescence based detection methods. A heterogeneous., sandwich immunoassay scheme was applied in both cases. The limit of detection of the immunofluorescence assays was 8 pM (1 ng/mL), while chemiluminescence allowed us to detect 424 fM (50 pg/mL) concentration of CRP in buffer. With approximate assay times of 30 min, the described microfluidic immunoassay approaches show great potential for rapid, but sensitive detection of disease markers at the point-of-care.

Immunoassays are some of the most crucial and versatile analytical tools and are widely used in the field of clinical diagnostics, forensics and biomolecular research. The assays are based on the highly specific and sensitive interactions of antibodies, produced by the immune system, with foreign molecules or antigens. Quantitative immunoassays are very useful in detecting small amounts of disease markers in physiological fluids, screening for infections or toxic substances, monitoring therapeutic drugs and screening for environmental contaminants. (Ekins, R., Nucl. Med. Biol. 1994, 21, 495-521; Brown, E. N., et al., Clin. Chem., 1996, 42, 893-903; Hatch, A., et al., Nat. Biotechnol. 2001, 19, 461-465) However, conventional immunoassays performed in microwell plates have several drawbacks, including long assay times, difficult fluid handling techniques, and high sample and reagent consumption, which have prevented immunoassay from being a point-of-care diagnostic tool. (Dodge, A., et al., Anal. Chem. 2001, 73, 3400-3409; Sato, K., et al., Anal. Chem. 2001, 73, 1213-1218; Gao, Y., et al., Proceedings of the 3rd International Conference on Microchannels and Minichannels, Toronto, 2005).

Recent advances in microfluidics and microfabrication technologies have lead to the development of “lab-on-a-chip” devices or μTAS (Micro Total Analytical Systems). Miniaturization of analytical processes offers the advantages of high-throughput assays, multistage automation and parallel processing of multiple analytes.(Zimmermann, M., et al., Biomed. Microdevices. 2005, 7(2), 99 110; Lin, F. Y. H., et al., The Analyst, 2004, 129(9), 823-828). With the microfluidic approach, the total assay time is considerably shortened and the sample/reagent consumption is lowered by virtue of the reduction in reaction chamber volume and increase in surface-to-volume ratio. The microfluidics-based “lab-on-a-chip” technology can therefore be applied to develop portable devices that can perform rapid and sensitive analysis of small volumes of diagnostic samples at the point-of-care.

In recent years, there has also been an increasing interest in saliva as a diagnostic medium. Analysis of saliva and other oral fluids has great potential in diagnosis of oral and systemic diseases, in preliminary screening for exposure to biological and chemical warfare agents and in monitoring for drugs. (Aguirre, A., et al., Cit. Rev. Oral Biol. Med. 1993, 4, 343-350; Christodoulides, N., et al., Lab Chip, 2005, 5, 261-269). Saliva is also an easier alternative to blood as a diagnostic fluid because of the non-invasive and convenient sample collection procedures. However, the use of salivary fluids for diagnosis is limited by the lack of high-sensitivity diagnostic methods that can detect the low concentrations of biomarkers expressed in saliva (Christodoulides, N., et al., Lab Chip, 2005, 5, 261-269).

In this work, we have developed a microfluidics-based immunoassay chip that can detect biomarkers at the levels of concentration expressed in saliva. C-reactive protein (CRP) was chosen as the model biomarker to assess the sensitivity of the methods. CRP is a 118 kD protein that is produced in the liver during episodes of acute inflammation or infection. It is classified as a characteristic acute phase reactant in human serum and a classic marker of inflammation (Kushner, I., et al., Clin. Rheumatol., 1994, 8, 513-530). Several studies have demonstrated the association between inflammation and cardiovascular disease (CVD) and testing of serum CRP levels is suggested as a new way of monitoring CVD risk. (Kriz, K., et al., Anal. Chem. 2005, 77, 5920-5924; Pearson, T. A., et al., Circulation, 2003, 107, 499-511; Ridker, P. M., et al., Cardiol. Clin. 2003, 21, 315-325) Clinical and epidemiological studies have also indicated that CVD may be associated with periodontitis and that systemic CRP may be a link between the two (Meurman, J. H., et al., Oral Surg. Oral Med. Oral Pathol. Oral Radiol. Endod. 2003, 96, 695-700; Wehrmacher, W. H. Dent. Today 2001, 20, 80-81). It has been shown that CRP biomarker can be detected in unstimulated whole saliva and its level is directly related to an individual's periodontal health (Christodoulides, N., et al., Lab Chip, 2005, 5, 261-269). However, to date no efficient and cost-effective method has been reported for rapid and sensitive detection of low, but pathophysiologically relevant concentrations of CRP, as needed for the development of a saliva-based point-of-care diagnostic technology. Current high-sensitivity CRP (hsCRP) testing kits designed for near-patient blood CRP analysis employ ELISA (Enzyme Linked Immunosorbent Assay) technique and have a limit of detection of 1.0 ng/mL (Christodoulides, N., et al., Lab Chip, 2005, 5, 261-269). The concentration range of CRP expressed in saliva is in pico- or femtomolar range, and cannot be detected by the above mentioned technique. At central hospital locations, hsCRP testing is performed utilizing turbidimetric or nephelometric homogeneous immunoassays on large clinical analyzers, (Kriz, K., et al., Anal. Chem. 2005, 77, 5920-5924) which cannot be easily translated into a point-of-care technology. We wanted to develop a new immunoassay technology platform that will enable on-site, high-sensitivity assays for detection of protein biomarkers.

We now show the development of-fluorescence and-chemiluminescence based microfluidic immunoassay methods for measuring low concentrations of CRP on a disposable plastic microchip. Microfluidics-based immunoassay methodologies have been previously developed in glass or PDMS (polydimethoxysiloxane) microchips (Dodge, A.; et al., Anal. Chem. 2001, 73, 3400-3409; Sato, K et al., Anal. Chem. 2001, 73, 1213-1218; Gao, Y. et al., Proceedings of the 3rd International Conference on Microchannels and Minichannels, Toronto, 2005). However, microfabricated chips made of glass are not ideal for disposable diagnostic applications as they entail high material and manufacturing costs, while PDMS lacks dimensional stability and has poor shelf-life. In this work, we used ZEONEX® (ZEONOR 690R, Zeon Chemicals Inc., Louisville, Ky.), a medical grade cyclic polyolefin to fabricate a plastic microfluidic chip. ZEONOR 690R exhibits very high light transmittance and low autofluorescence. The optical properties of ZEONEX are important for on-chip optical detection. Using the microfluidic immunoassay methodology described here, the complex bench-top diagnostic tests can be shrunk into a simple, hand-held device for detection of CRP in saliva at the point-of-care.

Materials. Cyclic polyolefin (ZEONEX 690R) was obtained as a gift from Zeon Chemicals Inc. (Louisville, Ky.). SU-8 50 photoepoxy and SU-8 developer were purchased from Microchem (Newton, Mass.). CRP antigen was purchased from Fitzgerald Industries International, Inc. (Concord, Mass.). IgG fraction of monoclonal rabbit anti-human CRP was purchased from EMD Biosciences (San Diego, Calif.) and IgG fraction of polyclonal goat anti-human CRP conjugated with FITC (fluorescein isothiocyanate) was purchased from Rockland Inc. (Gilbertsville, Pa.). IMMUN-STAR™ HRP Chemiluminescent Kit was obtained from Bio-Rad Laboratories, Inc. (Hercules, Calif.). Bovine Serum Albumin (98%, BSA) was purchased from Fisher Scientific (Fairlawn, N.J.). Protein A and FITC conjugated rabbit IgG were obtained from Sigma-Aldrich (St. Louis, Mo.) PEEK (Polyetheretherketone) capillaries of 360 μm o.d. and NANOPORT™ assemblies for chip-based fluidic connections were purchased from Upchurch Scientific (Oak Harbor, Wash.).

Chip Fabrication, Design and Operation. The microfluidic channels were fabricated by hot-embossing with an SU-8 master. Channels of 100 μm depth and 200 μm in width were fabricated by this method. The SU-8 masters were fabricated on piranha-cleaned silicon wafers by spinning SU-8 at a thickness of 100 μm onto the wafers. After pre-baking the wafers for 30 min at 95° C., the pattern was transferred through a mask by proximity contact lithography. This was followed by development with SU-8 developer and post-baking the wafers for 1.5 h at 175° C. After this fabrication process, SU-8 masters exhibited glassy mechanical properties and had the negative pattern of the channels. The wafers were then sputter coated with 500 Å of titanium for adhesion, followed by 1000 Å of aluminum. Sputter coating the master mold is an optional step. We found that the aluminum coating helped in the removal of the master from the substrate after the embossing is completed.

The microchannels were formed by hot-embossing with the master at 156° C. (20° C. above the T_{g} of ZEONEX 690R) and 250 psi for 5 minutes using a hot press (Heated Press 4386. Carver. Wabash, Ind.). The master and the substrate were manually separated at the de-embossing temperature, 126° C. (FIG. 1B). Wells of 1.5 mm diameter were drilled at the ends of the embossed microchannels to serve as solution reservoirs. To seal the channels, another piece of ZEONOR of the same dimensions was thermally bonded (136° C., 250 psi, 2 min.) on top in the hot press.

FIG. 2F shows a microchip with multiple channels. The reaction chamber consists of a 2 cm channel, which is connected to a sample introduction well and a collection well at opposite ends. All the channels have 200 μm (width)×100 μm (depth) cross-sections, so that the volume in the reaction chamber is 400 nL, making it a nanowell in functionality. With the hot-embossing method, channels with picoliter volumes can also be manufactured and applied as immunoassay reaction chambers. The antibodies and the antigens were introduced into the reaction channel at a flow rate of 100 μL/h with a KDS100 syringe pump (manufactured by KD Scientific, Holliston, Mass.). The syringe was connected to the microchip using PEEK tubing and NANOPORT™ fittings.

Immunofluorescence method. The fluorescence detection method was based on a heterogeneous sandwich assay scheme. In sandwich immunoassays, a monoclonal antibody specific to the target analyte (antigen), is bound to a surface. The sample fluid was contacted with the surface, whereby the antibody captures the target antigen. A labeled polyclonal antibody attaches to the antigen to complete the “sandwich”. The label (e.g., a linked enzyme or a fluorophore) generates a signal that is detected to quantify the captured antigen. Sandwich immunoassay is the most sensitive and specific immunoassay technique for antigen detection (Sato, K. et al., Anal. Chem. 2001, 73, 1213-1218); however, it is not desirable in a conventional immunoassay setup, because it involves many fluid handling steps for sample/reagent loading and washing. A microfluidic immunoassay method easily overcomes this drawback of the conventional method, because the reactions are controlled by simply pumping solutions in sequentially.

For the immunofluorescence assays, rabbit anti-human CRP antibody was used as the capture antibody and goat anti-human CRP antibody conjugated with FITC is used as the detection antibody. To enhance the sensitivity of the immunoassay reaction, a protein A layer was deposited on the channel walls prior to the immobilization of the capture antibodies. There is a major advantage of using the protein A layer prior to the attachment of the antibodies, because protein A has four high affinity binding sites for IgG (immunoglobulin G) of most species (Dodge, A., et al., Anal. Chem. 2001, 73, 3400-3409; Coen, M. C., et al., Coll. Int. Sci. 2001, 233, 180-189). If the antibodies are immobilized directly on the surface, they will bind in a random fashion and might not be oriented in the correct position to accept the target antigens. However, when an antibody binds to protein A, it is correctly aligned to receive the antigen (Dodge, A., et al., Anal. Chem. 2001, 73, 3400-3409). The bioactivity of adsorbed protein A was checked by immobilizing rabbit IgG conjugated with FITC (rIgG-FITC) to protein A. For this test, protein A at a concentration of 0.1 mg/mL was statically adsorbed on 2 cm diameter ZEONEX pieces for 30 min. 10 μg/mL of rIgG-FITC was then added to the protein A immobilized surface and allowed to bind for 30 min, after which the fluorescence was detected with a fluorescence microscope (Axiotech Materials Microscope, Carl Zeiss, Inc., Thornwood, N.Y.). A mercury lamp was used as the light source. Images were captured using an AXIOCAM MR CCD camera (Carl Zeiss, Inc.) and the images for processed using the AXIOVISION AC imaging software. The results were compared with the binding of rIgG-FITC to the native surface.

A schematic of the immunofluorescence reaction in the microchannel is shown in FIGS. 3B-3E. Protein A at a concentration of 0.1 mg/mL was physisorbed on the surface by pumping the solution into the channel at a flow rate of 100 μl/hour for 30 min and was allowed to incubate at room temperature for another 30 min. Following the immobilization of protein A, the rest of the channel surface was blocked with 1% BSA for 15 min to prevent non-specific adsorption of antibodies and antigens. The reaction channel was then washed twice by passing 5 μL of 1× PBS (phosphate buffered saline) by aspiration. A drop of solution was placed at the channel inlet and vacuum was applied at the other end by an aspiration tube. The washing buffer volume is 25 times the volume of the reaction channel, which ensures efficient washing. After the wash step, 10 μg/mL of the capture antibody was pumped through the channel for 3 min and incubated for 5 min at room temperature. Incubation as performed under a static condition when the flow was stopped. The reaction channel was washed twice with 1× PBS as mentioned earlier. CRP antigen at different concentrations was then passed though individual channels for 3 min, followed by 5 min of incubation and washed twice with 1× PBS. Following the incubation of the antigen, the detection antibody conjugated with FITC (1 μg/mL) was flowed through the channel for 3 min, incubated for 5 min. and washed with 1× PBS twice by aspiration. The fluorescence was then detected by the fluorescence microscope described earlier.

Chemiluminescence immunoassay method. Chemiluminescence is a highly sensitive technique with limits of detection in the low picogram range. In this work, chemiluminescence based immunoassay was performed with luminol as the substrate and horseradish peroxidase (HRP) as the enzyme conjugated to the secondary antibody. In the presence of hydrogen peroxide, HRP catalyzes the oxidation of luminol.

When oxidized luminol returns to its original state, an iridescent blue light is emitted, which can be detected by exposure to X-ray film, instant film, or an imager capable of detecting chemiluminescent signals. We used VERSADOC™ imaging system from Bio-Rad Laboratories, Inc. (Hercules, Calif.) to detect the chemiluminescent signals.

The chemiluminescent immunoassay schematic is shown in FIG. 3F. The channel surfaces were modified with protein A and 1% BSA as mentioned in the immunofluorescence technique. The steps of the immunoassay as used in this example are described in Table 1, where the primary and secondary antibodies are denoted as 1* Ab and 2° Ab respectively. After each incubation step, the channels were washed twice with 1× PBS by aspiration. The chemiluminescent signals were measured 2-3 min after the substrate was loaded into the channel. Table 1 below shows steps for chemiluminescence based immunoassay:

TABLE 1

| | | | Duration |
|---|---|---|---|
| No. | Step | Conc. | (min) |
| 1 | Load 1* Ab | 10 μg/mL | 3 |
| 2 | Incubate | — | 5 |
| 3 | Load antigen | 1-0.05 ng/mL | 3 |
| 4 | Incubate | — | 5 |
| 5 | Load 2° Ab | 1 μg/mL | 3 |
| 6 | Incubate | — | 5 |
| 7 | Load Anti-Rabbit IgG- | 0.1 μg/mL | 3 |
| | HRP | | |
| 8 | Incubate | — | 5 |
| 9 | Add chemiluminescent | — | — |
| | substrate | | |
| | Total | | 32 |

Bioactivity of Protein A. The fluorescent micrographs of immobilized IgG-FITC on a saturated protein A layer showed that the IgG were uniformly distributed over the surface and fluorescent intensity measured along a linear region of the sample was homogeneous (as shown in FIG. 5A). This confirmed that the protein A deposited on the Zeonex surface was non-denatured and was able to bind IgG specifically. On the other hand, the coverage of IgG on the native ZEONOR surface was inhomogeneous and the IgG formed isolated clusters over the surface. The fluorescent intensity profile along a linear region of the sample (FIG. 5B) showed inconsistent fluorescent peaks indicating that the non-specifically bonded IgG was unevenly distributed and there were regions on the surface where no IgG was deposited. It is also possible that the adsorbed IgG were washed away when the surfaces were rinsed with water before the fluorescence measurement (Coen, M. C., et al., Coll. Int. Sci. 2001, 233, 180-189).

Immunofluorescence. We took fluorescence images after the immunoassay. The CRP antigens could be reliably detected with the concentration of antigen in the range of 1 ng/mL to 500 ng/mL. Quantification of the fluorescence signals is plotted in FIG. 6. These results demonstrate the sensitivity of the immunofluorescence technique. The fluorescence signal obtained for a concentration of 0.5 ng/mL was not significantly different (ANOVA, p>0.05) from the signal obtained for the control (buffer with no antigen), indicating the limit of detection (LOD) with the fluorescence method is approximately 8 pM. The detection limit with this technique is far better than previously used magnetic permeability detection based immunoassay (LOD 0.2 mg/L), 12 and is at par with the commercially available “high-sensitivity” CRP (hsCRP) kits based on ELISA method (LOD 1 ng/mL) (Christodoulides, N., et al., Lab Chip, 2005, 5, 261-269).

Chemiluminescence results. We analyzed the chemiluminescence images for different concentrations of CRP and FIG. 7 shows the profile of the chemiluminescent signal intensity.

The limit of detection (LOD) of this assay in buffer was 50 pg/mL. Christodoulides et al. have shown that the levels of CRP in saliva of healthy individuals and periodontal disease patients are 92 pg/mL and 2001 pg/mL respectively (Lab Chip, 2005, 5, 261-269). It is clear that the chemiluminescence based immunoassay technique can detect CRP at concentration levels below that expressed in saliva. The detection limit is significantly better than the conventional calorimetric assays and the fluorescence immunoassay method.

The immunoassay methodologies developed here show excellent performance with respect to diagnostic sensitivity, speed and robustness. The limit of detection of the on-chip immunoassay via fluorescence was 8 pM (1 ng/ml concentration), while chemiluminescence allowed us to detect 424 fM (50 pg/mL concentration) of CRP in buffer. The immunofluorescence approach can be easily applied for detection of CRP biomarkers in serum or in saliva samples of diseased individuals, where generally higher concentrations of disease markers are expressed. Chemiluminescence is more sensitive approach and can be applied for screening saliva samples of either diseased or healthy individuals.

Chemiluminescence allows detection of biomarkers at concentration ranges below that expressed in saliva. Thus, it will be possible to dilute the saliva samples in buffer to lower the viscosity and heterogeneity associated with real-life saliva samples.

### Example 3

### Purification of Biomolecules

The DNA extraction efficiency of the monolith/silica columns was tested through spectroscopic measurement of absorption at 260 nm. The extraction procedure itself consisted of load, wash and elution steps. The loading solution consisted of 0.5 μg/mL of λ DNA in chaotropic buffer containing GuSCN (guanidium thiocyanate) and 3% BSA (Bovine Serum Albumin). 3% BSA was added to confirm that the separation column was able to separate nucleic acids in the presence of proteins. The microchannels were conditioned with the loading buffer (without DNA) for 5 min before the subsequent extraction in the channel. Then 75 μL of the loading solution was passed through the microchannel at a flow rate of 300 μL/hour with a KDS100 syringe pump (manufactured by KD Scientific, Holliston, Mass.). The syringe was connected to the microchip using 360-μm-i.d. PEEK tubing and NANOPORT™ fittings. 75 μL of the wash buffer consisting of 70% ethanol was then passed through the solid-phase. The proteins that were adsorbed onto the solid-phase during the load step were removed with the wash buffer, which was determined by absorbance measurements at 280 nm. Finally the DNA was eluted in water. The loaded microchannels were conditioned in the loading buffer (without DNA) for 5 min before the subsequent extraction in the channel. The loading solution, wash solution and the eluent were collected in microcuvettes and their concentrations were measured in a spectrophotometer (Eppendorf BioPhotometer, Eppendorf Scientific, Inc., Westbury, N.Y.).

To examine the binding of DNA onto the silica in the channel, fluorescence imaging was done with a fluorescent microscope (Axiotech Materials Microscope, Carl Zeiss, Inc., Thornwood, N.Y.). DNA stained with Hoechst 33258 dye was flowed through the solid phase at a flow rate of 300 μL/hour for 5 min in the presence of chaotropic buffer. The DNA retained in the solid-phase after the loading step was observed under the fluorescent microscope. The DNA was subsequently washed out of the channels with the elution buffer and the microchannels were checked under the fluorescent microscope again to visualize the elution efficiency. Residual fluorescence in the channels was indicative of incomplete elution.

The DNA extraction studies were performed on the monolith/silica system and demonstrated the effectiveness of the device for repetitive DNA extractions. The initial extraction efficiencies of the devices were found to be as high as 70%+3%, which is comparable with the sol-gel methods. FIG. 6 shows the percentage of loaded DNA eluted on three different chips. Extraction efficiency across different channels was relatively consistent; however, multiple extractions performed on the same microchannel were not very reproducible. The reduced efficiency was believed to be due to the breakdown of the monolith over time. It was observed that with multiple uses of the microchip, the mechanical instability of the monolith gradually lead to the collapsing of the monolithic nodules and pores, causing increased back pressure, unsteady flow patterns and drop in extraction efficiencies. Since the plastic chips are intended to be used as disposable devices, the stability of the system for repeated extractions was not of primary concern. Since the initial extraction efficiency was high and consistent, it seems to be a good solid-phase system for purification of nucleic acids. Future work will include increasing the overall efficiency of system by optimizing the flow rate and pH of the sample and elution buffer.

We showed that Hoechst stained DNA adsorbed strongly onto the silica embedded in the microchannel and bright fluorescence was observed in the channel. In contrast, very little fluorescence was seen in the channel after the DNA was eluted with water (FIG. 4), indicating good elution efficiency.

Electroosmotic flow and electrokinetic pumping. In preliminary testing, samples were flowed over columns using externally applied pressure. In a finished device, movement of the buffers over the solid-phase extraction column will be performed by electrokinetic pumping facilitated by electrodes inserted into the wells through the cover membranes.

Gel and capillary electrophoresis (CE) are used in conventional biology labs to separate biological molecules based on differences in charge and size. CE has been effectively miniaturized into microchannels made of many different materials. As long as the channel surface has a native charge, electroosmotic flow (EOF) can be generated by an applied field in the presence of an electrolyte. EOF is a unique feature of CE and is replicated in chip-based CE. The electrolyte charge separates at the walls of the capillary, creating a double layer. If the native surface charge is negative, the double layer is rich in cations, and an applied electric field results in the bulk flow of the electrolyte toward the negatively charged electrode. The strength of the surface charge and thus the velocity of the EOF are dependent on the channel material and the buffer system in use.

The mobility due to the EOF is: μₑₒ=(Eε ζ/4 πη), where η=viscosity, ζ=zeta potential (charge on capillary surface), E=the electric field and E is the dielectric constant of the buffer (or gel) system. The velocity of the electroosmotic flow, Veo, is: Veo=μeo (V/L), where V=the applied voltage and L=the length of the capillary. At the same time, electrophoretic flow is occurring. Positively charged biomolecules in the electrolyte are attracted toward the negative electrode, and negatively charged biomolecules move toward the positive electrode. At equilibrium, the force of the applied electric field on the charged particles is balanced by the frictional forces of drag on the particles moving through a viscous medium, and the electrophoretic mobility can be described by: μₑₚ=q/(6 πηr), where q=number of ionic charges, η=solution viscosity and r=ionic radius. These two mobilities determine the total mobility of a particular protein in a particular CE system. Since biomolecules are significantly larger than those of the buffer system, electrophoretic mobility is almost always slower than the EOF.

### Example 4

### Detection of Bacteria in a Sample

Isolation of bacterial nucleic acids. We have shows that we can successfully isolate nucleic acids from mammalian cells. Bacterial cell walls are much more robust and often require more vigorous lysing steps. The presence of the more robust bacterial cell walls also acts to plug an SPE column that has pores that are too small. Accordingly, we fabricated and tested a range of columns using different amounts of porogen as follows:

We obtained a non-toxigenic commercially available bacterial sample. We used C. difficile (ATCC, Manassas, Va.), as this organism represents one of the more difficult to diagnose infectious diarrheas. We cultured the sample at 37° C. in ATCC Medium No: 1053 Broth: Reinforced Clostridial Medium (Oxoid CM 149 or BD 218081) (ATCC, Manassas, Va.). We chemically lysed bacteria in an appropriate buffer system and produced serial dilutions of the crude cell lysate. We run the bacterial samples over micro-SPE columns, washed the columns, and extracted isolated nucleic acids. The isolated and concentrated nucleic acids from chips were removed and a polymerase chain reaction was performed using primers designed to detect the toxin A and B genes in the test organism. These are specific to the test organism chosen. In our test case we used primers designed to detect C. difficile toxins. The optimal micro-SPE columns for average pore size and silica content were chosen based on the elution efficiency of the test columns.

To detect a bacterial infection in the filed or at the bedside, it is necessary to be able to run samples from stool, throat cultures, saliva and urine to name a few examples. These samples are often far from ideal and may contain biomolecules and debris that can interfere with the operation and efficiency of the extraction column. The described example method can be used to optimize the chip for any type of biological material.

In addition, real samples contain many different bacterial species in addition to debris and other biological macromolecules including human cells and therefore human nucleic acids. Optimization of both the chip and the amount of sample loaded onto the chip may be necessary. It may be necessary to add agents to the sample introduction buffer that neutralizes specific contaminating components that interfere with the function of the column. These agents may be different depending on which body fluid is used as a sample for detection. A typical design for testing these specific conditions comprise mixing bacterial samples with well defined mixtures of lipids, proteins and carbohydrates; chemically lysing bacteria in an appropriate buffer system; producing serial dilutions of the crude cell lysate and running bacterial samples over micro-SPE columns; washing columns; extracting isolated nucleic acids; removing nucleic acids from chips; and running real time polymerase chain reactions using primers designed to detect the toxin A and B genes. Optionally, one can use the system described, supra to further optimize micro-SPE columns for average pore size and silica content.

Additionally, one may run the wash buffers and the isolated samples out on both standard and denaturing slab gels to assess the type and quantity of protein contaminants that are left behind. Gels can also be run to assess the quality of the isolated nucleic acids. Immunochemical techniques may be used to quantify the amount of lipids and carbohydrates in the wash buffers and the final eluted sample. Scanning electron microscopy may be performed on cross sections of the columns before and after separations in order to get a qualitative picture of whether or not the sample mixtures are clogging the columns or causing the internal structures to collapse.

Bacterial Detection Device Fabrication: The microfluidic channels are fabricated by hot-embossing with an SU-8 master. Channels of 100 μm depth and 100 μm/150 μm in width were fabricated by this method. The SU-8 masters were fabricated on piranha-cleaned silicon wafers by spinning SU-8 at a thickness of 100 μm onto the wafers. After pre-baking the wafers for 30 min at 95° C., the pattern is transferred through a mask by proximity contact lithography. This is followed by development with SU-8 developer and post-baking the wafers for 1.5 h at 175° C. After this fabrication process, SU-8 masters exhibit glass-like mechanical properties and have the negative pattern of the channels. The wafers were then sputter coated with 500 Å of titanium for adhesion, followed by 1000 Å of aluminum. Sputter coating the master mold is an optional step. We found that the aluminum coating helps in the removal of the master from the substrate after the embossing is completed.

The microchannels are formed by hot-embossing with the master at 90° C. (20° C. above the T_{g} of ZEONOR 750R) and 250 psi for 5 minutes using a hot press (Heated Press 4386, Carver, Wabash, Ind.). The master and the substrate are manually separated at the de-embossing temperature, 60° C. (FIG. 1B). To minimize artifacts that occur while removing the master, the separation is done when the plastic is no longer soft and deformable, but has not shrunk to a point where it is impossible to remove the master without causing substantial damage to the embossed substrate. Wells of 1.5 mm diameter are drilled at the ends of the embossed microchannels for sample introduction and collection. To seal the channels, another piece of ZEONOR of the same dimensions is thermally bonded (70° C., 250 psi, 2 min.) on top in the hot press.

Preparation of Solid-Phase. The fabricated channels were surface modified prior to the formation of the porous monolith in order to improve the adhesion of the monolith to the plastic device. Due to the relatively inert properties of the polymeric channel surfaces, it is difficult to achieve good bonding of the solid-phase with the native walls of the plastic devices. Hence the channel surfaces were modified via photografting with a thin interlayer polymer prior to the preparation of the monolith in the channel. The grafted layer allowed for covalent attachment of the monolith to the channel walls and prevented the formation of voids between the monolith and the channel surface. The grafted interlayer also stopped the monolith from migrating down the channel during separations.

Modification was achieved by using a benzophenone initiated surface photopolymerization process. The microchannels are filled with a 1:1 mixture of EDA and MMA with 3% benzophenone, which is a hydrogen abstracting photoinitiator. The chip was then UV-irradiated for 10 min at 254 nm UV wavelength and 200 mJ/cm2 energy in an ultraviolet exposure instrument (CL-1000 UV Crosslinker, UPV Inc., Upland, Calif.). The grafting step was carried out so that it led to very low conversion and avoided the formation of crosslinked polymer within the channels. The excess monomer was removed from the channels by rinsing with methanol at a flow rate of 100 μL/h for 1 h.

The monolith was formed by polymerization of a mixture of EDMA and BuMA monomers (Rohr, T., et al., Electrophoresis 22, 3959-67 (2001); Esch, M. B., Lab Chip 3, 121-7 (2003); Stachowiak, T. B., et al., Electrophoresis 24, 3689-93 (2003)). Porogenic solvents were added to the polymerization mixture to make the polymer monolith permeable. The porogenic solvents dissolve all the monomers and initiator to a form a homogeneous solution. The amount and type of porogen are controlled, and the phase separation process during polymerization leads to the desired pore structure (Yu, C., et al., Anal Chem 73, 5088-96 (2001)). A porogenic mixture of 1-dodecanol and cyclohexanol has been shown to be suitable for the preparation of porous monolithic columns using EDMA and BuMA as monomers. DMPAP was chosen as the UV initiator since it causes very fast polymerization, with complete conversion achieved within 10 min even at the lowest radiation power. The surface modified chips are filled with the mixture consisting of BuMA (24% wt), EDMA (16% wt), 1-dodecanol (42% wt), cyclohexanol (18% wt), DMPAP (1% wt with respect to monomers) and silica microbeads. The microchip is then irradiated with UV in the UV crosslinker at 200 mJ/cm2 for 2 min and then washed with methanol for 2 h at a flow rate of 100 μL/hour.

Collection and Preparation of Stool Samples. The cytotoxicity bioassay was considered the gold standard against which other cytotoxin assays are compared, given its high sensitivity (94-100%) and specificity (99%) (George, W. L., et al., J Clin Microbiol 15, 1049-53 (1982)). In this bioassay, stool is diluted with a buffer, centrifuged, filtered to remove bacteria and solids, and then placed in a cultured monolayer of fibroblasts. Both C. difficile toxins A and B, typically B more than A, however, disrupt the cytoskeleton and, when present at levels as low as a few molecules per cell, cause rounding. The specificity of this cytopathic effect is confirmed by preincubating a control sample with antibodies that neutralize C. difficile toxins. Cell rounding not thus blocked is referred to as “nonspecific cytotoxicity” which occurs in only ˜1% of samples. The bioassay is reported as “positive” or “negative.” Titers are not reported as they typically have no utility.

For the purposes of our study, all stool samples received by the BMC Microbiology laboratory for C. difficile cytotoxin bioassay will be processed in the standard manner and will in no way be altered. However, based on the size of the stool specimen and the volumes required for clinical testing, 1-5 ml of the residual supernatant following centrifugation will be removed, placed in a cryogenic tube, coded by date and time, and frozen at −80° C. in the Singh laboratory completely de-identified. The microbiology lab had a key to the samples and simply reported “positive” and “negative” for each sample as well as the incubation time from which the result was “called” (i.e. at the 4, 20, 48, or 72 hr inspection time). We were given a copy of the key that will have no patient information other than the age, sex and race/ethnicity of the person from whom the sample originated. The only result that is recorded is whether a coded sample in the freezer tested “positive,” “negative,” or “nonspecific,” and the time of incubation at which the call was made. Samples are then be thawed for nucleic acid extraction and molecular diagnostic testing as needed.

Estimation of Required Sample Size. Microfluidic devices for use in rapid C. difficile testing meet criteria for a valid new diagnostic test (Schoenfeld, P. et al. Gastroenterology 116, 1230-7 (1999)). There is a need for such a diagnostic in that there are no rapid “bedside” tests for C. difficile at the present time. The pre-test probability of a patient having C. difficile versus another cause of diarrhea does not obviate the need for this new diagnostic test. The new diagnostic test is compared to the gold standard of the cytotoxicity bioassay described above. We have set up a standard 2×2 table and calculated sample size to power sensitivity>specificity. We seek 90% sensitivity with a 95% confidence interval. We regard negative predictive value above positive predictive value as a false negative has graver consequences than a false positive result in C. difficile associated disease. The test is reproducible with minimal variation. Our patient population at BMC is fairly heterogeneous group. Therefore, our results are applicable to a wide general patient population.

Our calculations of power and sample size estimation uses a standard 2×2 table comparing the gold standard to our new diagnostic test (Jones, S. R., et al., Emerg Med J 20, 453-8 (2003)).

| | | Disease Presence by Gold Standard | |
|---|---|---|---|
| | | + | − |
| New Diagnostic | + | True Positive (TP) | False Negative (FN) |
| Test Result | − | False Positive (FP) | True Negative (TN) |

Sensitivity refers to the proportion of people with disease who have a positive test result: Sensitivity=TP÷(TP+FP). Specificity refers to the proportion of people without disease who have a negative test result: Specificity=TN÷(FP+TN). The Likelihood Ratio (LR) is the likelihood that a given test result would be expected in a patient with the target disorder compared to the likelihood that that same result would be expected in a patient without the target disorder. The likelihood ratio for a positive test result (LR+)=sensitivity÷(1 specificity); similarly, the likelihood ratio for a negative test result (LR−)=(1−sensitivity)÷specificity.

Our calculation for the sample size needed for a sensitivity of 90% is as follows:

TP+FN=(z²×(Sensitivity(1−Sensitivity))÷(Confidence Interval)². In our case, z=1.962, sensitivity=0.90, confidence interval=0.05, prevalence=0.10. Thus, with our stated target parameters: TP+FN=1.9622×(0.9(1−0.9))/0.052=138.3. N_{sensitivity}=TP+FN/Prevalence. Thus, N_{sensitivity}=138.3/0.10=1383.

Thus, we would need to test 1383 consecutive stool samples in a prospective study to characterize a diagnostic test that had a 90% specificity. However, during the instrument development phase, we can retrospectively weight the prevalence of positive samples based on stool cytotoxicity assay results, the gold standard. Thus, we set the prevalence of C. difficile positive stools at 0.5, we require a minimum of 277 samples.

1. A microfluidic device comprising:
(a) a substrate that is not glass with at least one channel of less than 150 μm in diameter, wherein the channel has an inlet, an outlet, and an internal space with a surface between the inlet and the outlet;
(b) a first porous polymer monolith comprising a first monomer within the internal space, wherein the porous polymer monolith comprises a second monomer, and is attached to said first polymer in at least one region of the internal space, wherein the first and the second monomers may be of the same or different material; and
(c) a second porous polymer monolith impregnated with particles within said internal space.

2. The microfluidic device of claim 1, wherein the channel has at least one section with a serpentine-shaped channel between the inlet and the outlet.

3. The microfluidic device of claim 1, wherein the particles are silica particles.

4. The microfluidic device of claim 1, wherein the particles are nanotubes of 1-20 microns in length and 30-150 nm in diameter.

5. The microfluidic device of claim 1, wherein the particles comprise a mixture of silica particles and nanotubes.

6. The microfluidic device of claim 1, wherein the substrate comprises polyolefin.

7. The microfluidic device of claim 1, wherein the first polymer comprises polyvinyl monomer.

8. The microfluidic device of claim 1, wherein the second polymer comprises poly-vinyl monomer.

9. The microfluidic device of claim 1, wherein the surface of the internal space further comprises protein A.

10. A method for manufacturing a microfluidic channel in a microfluidic device, comprising:
(a) providing a substrate having at least one channel disposed thereupon;
(b) filling the at least one channel with a first monomer solution comprising a photoinitiator and a monomer;
(c) exposing the solution to ultraviolet light for polymerizing said solution to a predetermined degree to form a polymer layer grafted to the wall of said channel;
(d) removing ungrafted monomer from the channel;
(e) filling the channel provided with the grafted polymer layer with a second monomer mixture impregnated with particles including a photinitiator for formation of a porous polymer monolith; and
(f) exposing the second monomer mixture to ultraviolet light for polymerizing said second monomer mixture to form a porous polymer monolith attached to the wall of said channel through the grafted polymer layer.

11. The method of claim 10, wherein the particles are silica particles.

12. The method of claim 10, wherein the particles are nanotubes.

13. The method of claim 10, wherein the particles comprise a mixture of silica particles and nanotubes.

14. The method of claim 10, wherein the substrate comprises polyolefin.

15. The method of claim 10, wherein the first polymer comprises polyvinyl monomer.

16. The method of claim 10, wherein the second polymer comprises polyvinyl monomer.

17. The method of claim 10, wherein the surface of the internal space of the channel is further modified with protein A.

18. A method of isolating biomolecules from a sample using a microfluidic device of claim 1 comprising the steps of:
(a) adding a sample through the inlet to the internal space of the channel of claim 1;
(b) applying at least one cell lysis buffer through the inlet to the channel;
(c) applying at least one washing buffer through the channel; and
(d) applying at least one elution buffer through the channel, wherein the elution buffer elutes the biomolecule from the internal channel through the outlet.

19. A method of identifying a biomolecule in a sample using the microfluidic device of claim 1 comprising the steps of:
(a) adding a sample through the inlet to the internal space of the channel of claim 1;
(b) applying at least one cell lysis buffer through the channel;
(c) applying at least one washing buffer through the channel; and
(d) identifying the biomolecule.

20. The method of claim 19, wherein identification of the biomolecule is performed inside the channel of the microfluidic device.

21. The method of claim 19, wherein the method further comprises a step of eluting the sample, and wherein the identification of the sample is performed outside the channel of the microfluidic device.

22. The method of claim 19, wherein the identification of the biomolecule comprises using polymerase chain reaction (PCR) inside the microfluidic device by adding a mixture of buffer, oligonucleotide primers, polymerase erase and nucleotides through the inlet into the at least one channel and placing the microfluidic device into a thermocycler.

23. The method of claim 19, wherein the internal surface of the microfluidic device is modified with protein A.

24. The method of claim 23, wherein the internal surface of the microfluidic device is further modified by attaching a first antigen into the internal surface of the channel, wherein the first antibody is capable of binding to an antigen present in the biomolecule.

25. The method of claim 24, further comprising attaching a second antibody to the surface of the internal space of the channel of the microfluidic device, wherein the second antibody is labeled and capable of recognizing the first antibody that is bound to the antigen present in the biomolecule and detecting the labeled second antibody, wherein the presence of the label is indicative of presence of the biomolecule in the sample.

26. The method of claim 18, wherein the sample is suspected to contain a disease causing agent carrying a detectable biomolecule.

27. The method of claim 26, wherein the disease causing agent is Clostridium difficile.
